(19) United States
(12) Patent Application Publication
(10) Pub. No.: US 2026/0179721 A1
(43) Pub. Date: Jun. 25, 2026

(54) METHODS AND SYSTEMS FOR CREATING METHYLATION-BASED SCORES FOR CANCER PREDICTION AND TUMOR TISSUE OF ORIGIN DETERMINATION

(71) Applicant: Foundation Medicine, Inc., Boston, MA (US)

(72) Inventors: Daniel LIEBER, Needham, MA (US); Neil PETERMAN, San Francisco, CA (US); Justin NEWBERG, Cambridge, MA (US)

(73) Assignee: Foundation Medicine, Inc., Boston, MA (US)

(21) Appl. No.: 19/427,310

(22) Filed: Dec. 19, 2025

Related U.S. Application Data
(60) Provisional application No. 63/737,469, filed on Dec. 20, 2024.

(51) Int. Cl. G16B 20/00; G16B 40/20; G16H 50/20

(52) U.S. Cl. CPC G16B 20/00; G16B 40/20; G16H 50/20

(57) ABSTRACT

Methods for generating and evaluation methylation meta scores and their use for the detection of disease and prediction of tumor tissue of origin are described. The methods may comprise, e.g., generating annotated genomic interval data comprising at least one of: (i) genomic coordinates, and (ii) associated genomic annotation data; providing annotated patient sample data comprising at least one of: (i) methylation sequencing data, and (ii) associated clinical annotation data; generating an aggregated methylation meta score matrix comprising candidate methylation meta scores; inputting (i) the aggregated methylation meta score matrix, and (ii) the associated clinical annotation data for at least a subset of patient samples as training data for training a machine learning model; and training the model using the training data to identify an optimal methylation meta score for disease detection and/or prediction of disease tissue of origin (TOO) for at least one disease type based on methylation sequencing data.

### CROSS-REFERENCE TO RELATED APPLICATIONS

This application claims priority benefit to U.S. Provisional Application No. 63/737,469, filed Dec. 20, 2024, the entire contents of which is incorporated herein by reference for all purposes.

### FIELD

The present disclosure relates generally to methods and systems for analyzing genomic profiling data, and more specifically to methods and systems for creating and using methylation-based scores for cancer prediction and tumor tissue of origin determination.

### BACKGROUND

Methylation sequencing to identify DNA methylation sites has become increasingly important as a tool for studying the epigenetic mechanisms that regulate gene expression and the abnormal methylation patterns that can be indicative of some types of disease. Because DNA methylation patterns are complex and can vary with, for example, subject age, tissue type, and environmental factors, as well as with disease state, there is a need for improved methylation-based metrics that can serve as diagnostic and/or prognostic biomarkers for disease.

### BRIEF SUMMARY OF THE INVENTION

Disclosed herein are machine learning-based methods and systems for detection of disease and/or prediction of disease tissue of origin (TOO) based on methylation meta scores, where a methylation meta score is a mathematical combination of weighted methylation scores assigned to each of a plurality of genomic intervals that have been identified to be of relevance for a specified disease state. The disclosed methods enable the determination of methylation-based scores for detecting disease (e.g., cancer) and/or determining tissue of origin (TOO) (e.g., tumor tissue of origin (TTOO)), where a given methylation-based score has been optimized for detecting and/or determining tissue of origin for a specific disease.

Disclosed herein are methods for detection of disease and/or prediction of disease tissue of origin (TOO), the methods comprising: generating, using one or more processors, annotated genomic interval data comprising at least one of: (i) genomic coordinates, and (ii) associated genomic annotation data for a plurality of candidate genomic intervals; providing, using the one or more processors, annotated patient sample data comprising at least one of: (i) methylation sequencing data, and (ii) associated clinical annotation data for a plurality of patient samples; generating, using the one or more processors, an aggregated methylation meta score matrix comprising candidate methylation meta scores; inputting, using the one or more processors, (i) the aggregated methylation meta score matrix, and (ii) the associated clinical annotation data for at least a subset of the plurality of patient samples as training data for training a machine learning model; and training, using the one or more processors, the machine learning model using the training data to identify an optimal methylation meta score for disease detection and/or prediction of disease tissue of origin (TOO) for at least one disease type based on methylation sequencing data.

In some embodiments, the method further comprises providing methylation sequencing data derived from a sample from a subject as input to the trained machine learning model to detect a presence of a disease and/or predict a disease TOO in the subject. In some embodiments, the annotated genomic interval data comprises a Browser Extensible Data (BED) file. In some embodiments, the genomic annotation data comprises gene or partial gene sequence identification, promoter sequence coordinates, regulatory sequence coordinates, enhancer sequence coordinates, transcription start sites, open chromatin regions, known CpG cluster locations, known aberrant methylation pattern locations, or any combination thereof for each candidate genomic interval of the plurality.

In some embodiments, the candidate methylation meta scores in the aggregated methylation meta score matrix are based on mathematical combinations of weighted methylation scores assigned to each of the plurality of candidate genomic intervals.

In some embodiments, generating the aggregated methylation meta score matrix comprises: generating, using the one or more processors, a first matrix, wherein a given cell in the first matrix comprises a methylation score calculated for a corresponding candidate genomic interval based on methylation sequencing data for a corresponding patient sample; generating, using the one or more processors, a second matrix, wherein a given cell in the second matrix comprises a weighted methylation score assigned to a given candidate genomic interval of the plurality of candidate genomic intervals that indicates how informative the given candidate genomic interval is for disease detection and/or prediction of disease tissue of origin (TOO) in at least a subset of the plurality of patient samples; and combining, using the one or more processors, the first matrix and the second matrix to generate the aggregated methylation meta score matrix. In some embodiments, the first matrix comprises a number of rows equal to a total number of patient samples in the plurality of patient samples and a number of columns equal to a total number of candidate genomic intervals in the plurality of candidate genomic intervals. In some embodiments, the second matrix comprises a number of rows equal to a total number of candidate genomic intervals in the plurality of candidate genomic intervals and a number of columns equal to a user-defined number of candidate methylation meta scores.

In some embodiments, the aggregated methylation meta score matrix comprises a number of rows equal to the total number of candidate genomic intervals in the plurality of candidate genomic intervals and a number of columns equal to a user-defined number of candidate methylation meta scores.

In some embodiments, a given cell in the aggregated methylation meta score matrix comprises a candidate methylation meta score based on a mathematical combination of the weighted methylation scores in the second matrix.

In some embodiments, training the machine learning model comprises using a cross-validation training procedure.

In some embodiments, the methylation sequencing data comprises methylation sequencing data derived from a targeted sequencing method, a whole exome sequencing (WES) method, or a whole genome sequencing (WGS) method. In some embodiments, the methylation sequencing data comprises data obtained by sequencing nucleic acid molecules that have been subjected to a cytosine conversion reaction. In some embodiments, the cytosine conversion reaction comprises a bisulfite conversion reaction or an enzymatic conversion reaction.

In some embodiments, the clinical annotation data comprises patient gender, age, smoking status, disease type, disease stage, sample type, lab protocol for sample collection and processing, lab protocol for performing methylation sequencing, clinical data for treatment outcomes, or any combination thereof.

In some embodiments, the methylation score comprises a percent fully methylated score or a percent fully unmethylated score.

In some embodiments, a high value for the weighted methylation score assigned to each candidate genomic interval indicates that the candidate genomic interval is informative for disease detection and/or prediction of disease tissue of origin (TOO). In some embodiments, a low value for the weighted methylation score assigned to each candidate genomic interval indicates that the candidate genomic interval is not informative for disease detection and/or prediction of disease tissue of origin (TOO).

In some embodiments, the weighted methylation score assigned to each candidate genomic interval of the plurality of candidate genomic intervals is calculated based on a comparison of a methylation metric for each candidate genomic interval for a first subset of the plurality of patient samples to that for each candidate genomic interval for a second subset of the plurality of patient samples. In some embodiments, the methylation metric comprises a CCMF signal-to-noise ratio calculated for each candidate genomic interval for the first and second subsets of the plurality of patient samples. In some embodiments, the first subset of the plurality of patient samples comprises patient samples from patients diagnosed with a first disease. In some embodiments, the second subset of the plurality of patient samples comprises control samples from patients diagnosed with the first disease. In some embodiments, the second subset of the plurality of patient samples comprises patient samples from patients diagnosed with a second disease.

In some embodiments, the method further comprises selecting a subset of the plurality of candidate genomic intervals based on the weighted methylation score assigned to each candidate genomic interval and modifying the second matrix to reduce the number of rows accordingly.

In some embodiments, the method further comprises using the identified optimal methylation meta score to detect disease in a subject based on methylation sequencing data derived from a sample from the subject.

In some embodiments, the method further comprises using the identified optimal methylation meta score to predict a disease tissue of origin (TOO) in a subject based on methylation sequencing data derived from a sample from the subject.

In some embodiments, the method further comprises using the identified optimal methylation meta score to select a disease treatment for a subject based on methylation sequencing data derived from a sample from the subject.

In some embodiments, the method further comprises using the identified optimal methylation meta score to predict a disease treatment outcome for a subject based on methylation sequencing data derived from a sample from the subject.

In some embodiments, the method further comprises using the identified optimal methylation meta score to identify a subject for inclusion in a clinical trial for a disease treatment based on methylation sequencing data derived from a sample from the subject.

In some embodiments, herein the disease is a cancer or a genetic disorder. In some embodiments, the disease is cancer, and the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the disease is a cancer, and the disease treatment comprises an anti-cancer therapy. In some embodiments, the anti-cancer therapy comprises chemotherapy, radiation therapy, immunotherapy, a targeted therapy, an neoantigen-based therapy, or surgery.

In some embodiments, the plurality of patient samples comprises tissue biopsy samples, liquid biopsy samples, or a combination thereof. In some embodiments, the plurality of patient samples comprises liquid biopsy samples, and the liquid biopsy samples comprise blood, plasma, cerebrospinal fluid, sputum, stool, urine, or saliva samples.

Disclosed herein are methods for detection of disease and/or prediction of disease tissue of origin (TOO), the methods comprising: receiving, at one or more processors, methylation sequencing data derived from a sample from a subject; determining, using the one or more processors, a methylation score for each genomic interval in a predetermined set of genomic intervals represented in the methylation sequencing data; providing, using the one or more processors, the methylation scores for the predetermined set of genomic intervals as input to a trained machine learning model configured to predict a presence of disease and/or a disease tissue of origin (TOO) based on a methylation meta score derived from the methylation scores for the predetermined set of genomic intervals; and outputting, using the one or more processors, a prediction of a presence of disease and/or a disease tissue of origin (TOO) for the subject.

In some embodiments, the methylation sequencing data is generated from one or more sequencing reads associated with the sample. In some embodiments, the methylation score comprises a methylation fraction score, a Cluster Consensus Methylation Fraction (CCMF) score, an unmethylated fraction score, or a Cluster Consensus Unmethylated Fraction (CCUF) score. In some embodiments, he predetermined set of genomic intervals is identified based on a comparison of the methylation score calculated for each genomic interval represented in the methylation sequencing data to a methylation score threshold. In some embodiments, the predetermined set of genomic intervals comprises all genomic intervals for which the calculated methylation score is greater than or equal to the methylation score threshold. In some embodiments, the methylation score threshold is disease-specific. In some embodiments, the methylation score threshold is disease-independent.

In some embodiments, the methylation meta score comprises an algebraic combination of the methylation scores determined for the predetermined set of genomic intervals and/or tree-based models to determine methylation scores for specific genomic intervals and/or a number of sequence reads that map to each genomic interval of the predetermined set of genomic intervals.

In some embodiments, the machine learning model is trained on a training date set comprising methylation scores determined for a plurality of genomic intervals represented in methylation sequencing data for a first cohort of subjects diagnosed with a first disease. In some embodiments, the training data set further comprises methylation scores determined for the plurality of genomic intervals represented in methylation sequencing data for non-diseased control samples from the first cohort of subjects. In some embodiments, the non-diseased control samples are of a different sample type than diseased samples used to generate the methylation sequencing data for the first cohort of subjects. In some embodiments, the training data further comprises methylation scores determined for the plurality of genomic intervals represented in methylation sequencing data for a second cohort of subjects diagnosed with a second disease. In some embodiments, the training data set further comprises methylation scores determined for the plurality of genomic intervals represented in methylation sequencing data for non-diseased control samples from the second cohort of subjects. In some embodiments, the non-diseased control samples are of a different sample type than diseased samples used to generate the methylation sequencing data for the second cohort of subjects. In some embodiments, the methylation sequencing data for the second cohort of subjects is derived from a different sample type than that for the first cohort of subjects. In some embodiments, the trained machine learning model is configured to predict a presence of the first disease, a presence of the second disease, a disease TOO for the first disease, and/or a disease TOO for the second disease based on a methylation meta score derived from the methylation scores for the predetermined set of genomic intervals

In some embodiments, the disease is cancer or a genetic disorder. In some embodiments, the disease is cancer, and the cancer is colorectal cancer (CRC) or lung cancer. In some embodiments, the first disease is colorectal cancer (CRC) and the second disease is lung cancer.

In some embodiments, the trained machine learning model is configured to output the methylation meta score, and wherein the method further comprises using the methylation meta score to select a disease treatment for the subject.

In some embodiments, the trained machine learning model is configured to output the methylation meta score, and wherein the method further comprises using the methylation meta score to predict a disease treatment outcome for the subject.

In some embodiments, the trained machine learning model is configured to output the methylation meta score, and wherein the method further comprises using the methylation meta score to identify the subject for inclusion in a clinical trial for a disease treatment.

In some embodiments, the methylation sequencing data comprises methylation sequencing data derived from a targeted sequencing method, a whole exome sequencing (WES) method, or a whole genome sequencing (WGS) method. In some embodiments, the methylation sequencing data comprises bisulfite sequencing data, enzymatic methyl (EM) sequencing data, or a combination thereof.

In some embodiments, the sample from the subject is a tissue biopsy sample or a liquid biopsy sample. In some embodiments, the sample from the subject is a liquid biopsy sample, and the liquid biopsy sample is a blood, plasma, cerebrospinal fluid, sputum, stool, urine, or saliva sample.

Disclosed herein are systems comprising: one or more processors; and a memory communicatively coupled to the one or more processors and configured to store instructions that, when executed by the one or more processors, cause the system to perform any of the methods described herein.

Also disclosed herein are non-transitory computer-readable storage media storing one or more programs, the one or more programs comprising instructions, which when executed by one or more processors of a system, cause the system to perform any of the methods described herein.

Disclosed herein are methods for detection of disease, prediction of disease tissue of origin (TOO), and/or calculation of a tumor quantity metric, the methods comprising: generating, using one or more processors, annotated genomic interval data comprising at least one of: (i) genomic coordinates, and (ii) associated genomic annotation data for a plurality of candidate genomic intervals; providing, using the one or more processors, annotated patient sample data comprising at least one of: (i) methylation sequencing data, and (ii) associated clinical annotation data for a plurality of patient samples; generating, using the one or more processors, an aggregated methylation meta score matrix comprising candidate methylation meta scores; inputting, using the one or more processors, (i) the aggregated methylation meta score matrix, and (ii) the associated clinical annotation data for at least a subset of the plurality of patient samples as training data for training a machine learning model; and training, using the one or more processors, the machine learning model using the training data to identify an optimal methylation meta score for disease detection, prediction of disease tissue of origin (TOO) for at least one disease type, and/or calculation of a tumor quantity metric based on methylation sequencing data. In some embodiments, the tumor quantity metric is a tumor fraction.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flowchart for identifying an optimal methylation meta score for disease detection and/or prediction of disease tissue of origin, in accordance with one implementation of the disclosed methods.

FIG. 2 provides a non-limiting example of a process flowchart for predicting a presence of disease and/or a disease tissue of origin for a subject, in accordance with one implementation of the disclosed methods.

FIG. 3 depicts an exemplary computing device or system in accordance with one embodiment of the present disclosure.

FIG. 4 depicts an exemplary computer system or computer network, in accordance with some instances of the systems described herein.

FIG. 5 provides a non-limiting schematic illustration of a process for generating candidate methylation meta scores and identifying an optimal methylation meta score for cancer-specific disease detection and tumor tissue of origin prediction.

FIG. 6 provides a non-limiting schematic illustration of the calculation of Cluster Consensus Methylation Fraction (CCMF).

FIG. 7 provides a non-limiting example of data illustrating the challenges of using a methylation score-based assay to discriminate between cancerous and healthy tissue samples.

FIG. 8 provides a non-limiting example of methylation data for colorectal cancer (CRC) and lung cancer patients, and illustrates the selection of genomic intervals for generating methylation meta scores for the detection of CRC and lung cancer that differentiate between these cancer types.

FIG. 9 provides a non-limiting schematic illustration of the training data used to train a machine learning model and identify an optimal methylation meta score for cancer prediction, and the methods used to assess prediction performance.

FIGS. 10A-B provides a non-limiting example of receiver operator characteristic (ROC) curves for meta score-based machine learning models used for lung cancer detection (FIG. 10A) and tumor tissue of origin classification (FIG. 10B).

### DETAILED DESCRIPTION

Machine learning-based methods and systems for detection of disease (e.g., cancer) and/or prediction of disease tissue of origin (TOO) (e.g., tumor tissue of origin (TTOO)) are described. The methods are based on the determination of optimal, disease-specific methylation meta scores, where a methylation meta score is a mathematical combination of weighted methylation scores assigned to each of a plurality of genomic intervals that have been identified to be of relevance for the specified disease state.

In some instances, for example, methods (e.g., computer-implemented methods) are described that comprise generating annotated genomic interval data comprising at least one of: (i) genomic coordinates, and (ii) associated genomic annotation data for a plurality of candidate genomic intervals; providing (or generating) annotated patient sample data comprising at least one of: (i) methylation sequencing data, and (ii) associated clinical annotation data for a plurality of patient samples; generating an aggregated methylation meta score matrix comprising candidate methylation meta scores; inputting (i) the aggregated methylation meta score matrix, and (ii) the associated clinical annotation data for at least the subset of the plurality of patient samples as training data for training a machine learning model; and training the machine learning model using the training data to identify an optimal methylation meta score for disease detection and/or prediction of disease tissue of origin (TOO) for at least one disease type based on methylation sequencing data.

In some instances, the methods further comprise providing methylation sequencing data derived from a sample from a subject as input to the trained machine learning model to detect a presence of a disease and/or predict a disease TOO in the subject.

In some instances, the candidate methylation meta scores in the aggregated methylation meta score matrix are based on mathematical combinations of weighted methylation scores assigned to each of the plurality of candidate genomic intervals.

In some instances, generating the aggregated methylation meta score matrix comprises: generating a first matrix, where a given cell in the first matrix comprises a methylation score calculated for a corresponding candidate genomic interval based on methylation sequencing data for a corresponding patient sample; generating a second matrix, wherein a given cell in the second matrix comprises a weighted methylation score assigned to a given candidate genomic interval of the plurality of candidate genomic intervals that indicates how informative the given candidate genomic interval is for disease detection and/or prediction of disease tissue of origin (TOO) in at least a subset of the plurality of patient samples; and combining the first matrix and the second matrix to generate the aggregated methylation meta score matrix.

In some instances, methods (e.g., computer-implemented methods) are described that comprise receiving methylation sequencing data derived from a sample from a subject; determining a methylation score for each genomic interval in a predetermined set of genomic intervals represented in the methylation sequencing data; providing the methylation scores for the predetermined set of genomic intervals as input to a trained machine learning model configured to predict a presence of disease, predict a disease tissue of origin (TOO), and/or predict a tumor fraction for a sample based on a methylation meta score derived from the methylation scores for the predetermined set of genomic intervals; and outputting a prediction of a presence of disease, a prediction of a disease tissue of origin (TOO) for the subject, and/or a prediction of a tumor fraction of a sample from the subject.

In some instances, the methylation score may comprise a score based on methylation features associated with an individual genomic interval. In some instances, the methylation score may comprise a score based methylation features associated with one or more genomic intervals (e.g., a set of genomic intervals). In some instances, for example, the methylation score may comprise a methylation fraction score, a Cluster Consensus Methylation Fraction (CCMF) score, an unmethylated fraction score, or a Cluster Consensus Unmethylated Fraction (CCUF) score. In some instances, a methylation meta score may be derived from a set of methylation scores, each of which is associated with one or more genomic intervals.

In some instances, the predetermined set of genomic intervals is identified based on a comparison of the methylation score calculated for each genomic interval represented in the methylation sequencing data to a methylation score threshold. In some instances, the predetermined set of genomic intervals comprises all genomic intervals for which the calculated methylation score is greater than or equal to the methylation score threshold. In some instances, the methylation score threshold is disease-specific. In some instances, the methylation score threshold is disease-independent.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Creation of Methylation-Based Meta Scores for Cancer Prediction and Tumor Tissue of Origin Determination

The disclosed methods have been developed as part of an ongoing effort to identify improved approaches for accurately predicting cancer status and tumor type from plasma samples of people at average or high risk of cancer. The machine learning-based methods described herein provide a way to identify cancer and tumor tissue of origin signals in methylation data, and to generate improved methylation “meta” scores associated with particular cancer types or subtypes for use in disease detection and tissue of origin (TOO) determination, where each methylation meta-score encompasses methylation data from a number of genomic intervals and is derived from methylation sequencing data for both cancer and non-cancer samples.

The disclosed methods constitute a single algorithmic workflow that can be used to generate methylation meta scores (e.g., algebraic combinations of methylation scores for specific genomic intervals and/or tree-based models to determine methylation scores for specific genomic intervals) that are specific to one or more types (or subtypes) of cancer, while also exploring various parameters (such as how many and which genomic loci to include) used to optimize the methylation meta scores. The methylation meta score can be made specific to the intersection or non-intersection of two or more cancer types (e.g. specific to lung cancer but not colorectal cancer (CRC), or specific to lung cancer and CRC).

The algorithmic workflow can utilize a variety of different methylation metrics, and is scalable and generalizable to cover different disease types and subtypes (e.g. lung and CRC, and also subtypes of each, or other cancer types such as pancreas, liver, etc.). The approach can be applied to a variety of different applications (e.g., cancer detection, tissue of origin classification, non-cancer anomaly detection (e.g., detection of rare genetic conditions that cause methylation changes in the absence of cancer), etc.).

A key advantage of the disclosed methods is that many different candidate methylation meta scores can be generated, and those that are the most useful for predicting cancer and determining tissue of origin (for specific cancer types) can then be identified as part of training an appropriate machine learning model. The approach has been tested on, e.g., lung cancer and CRC, and methylation meta scores based on methylation data for specific genomic regions identified as relevant for lung cancer regions minus those identified as relevant for CRC, and vice versa, worked remarkably well for predicting the tumor tissue of origin.

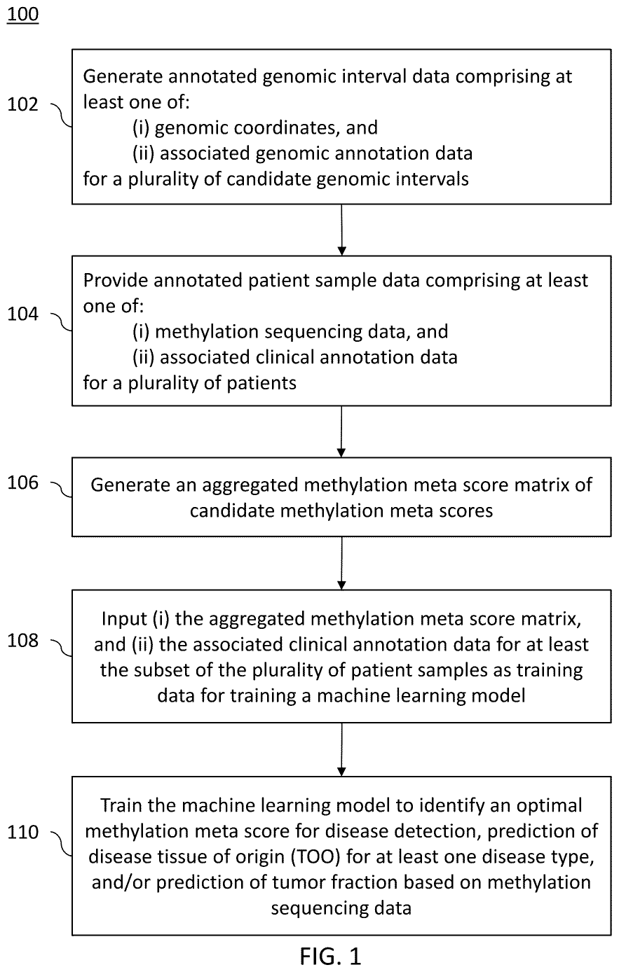

FIG. 1 provides a non-limiting example of a flowchart for a process 100 for identifying an optimal methylation meta score for disease detection and/or prediction of disease tissue of origin. Process 100 can be, for example, a computer-implemented method, and can performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, annotated genomic interval data is generated, where the annotated genomic interval data comprises at least one of: (i) the genomic coordinates, and (ii) associated genomic annotation data for a plurality of candidate genomic intervals. In some embodiments, the associated genomic annotation data is genomic annotation data associated with candidate genomic intervals of for a plurality of candidate genomic intervals.

In some instances, for example, the annotated genomic interval data may comprise a Browser Extensible Data (BED) file of genomic intervals to analyze, where the BED file is a text file used to store a list of genomic intervals (or genomic regions) as a list of genomic coordinates and associated genomic annotation data. Examples of genomic annotation data include, but are not limited to, gene or partial gene sequence identification, promoter sequence coordinates, regulatory sequence coordinates, enhancer sequence coordinates, transcription start sites, open chromatin regions, known CpG cluster locations, known aberrant methylation pattern locations, or any combination thereof.

In some instances, a candidate genomic interval may comprise a CpG cluster, a short genomic interval containing multiple CpG sites (i.e., sites comprising a cytosine followed by guanine in the 5′ to 3′ direction, where the cytosine residue is often methylated in mammalian DNA) that starts and ends with CG in the reference sequence. In some instances, the length of a CpG cluster may range from about 10 to 300 bp (e.g., about 10, 20, 30, 40, 50, 60, 70, 80, 90, 100, 120, 140, 160, 180, 200, 220, 240, 260, 280, or 300 bp, or any value within this range). In some instances, the length of a CpG cluster may range from about 30 to 150 bp (e.g., about 30, 40, 50, 60, 70, 80, 90, 100, 110, 120, 130, 140, or 150 bp, or any value within this range). In some instances, the length of a CpG cluster may range from about 40 to 100 bp (e.g., about 40, 50, 60, 70, 80, 90, or 100 bp, or any value within this range.

In some instances, the plurality of candidate genomic intervals may comprise between about 10 and about 100,000 candidate genomic intervals. In some instances, the plurality of candidate genomic intervals may comprise at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1,000, at least 5,000, at least 10,000, at least 20,000, at least 30,000, at least 40,000, at least 50,000, at least 60,000, at least 70,000, at least 80,000, at least 90,000, at least 100,000, or more than 100,000 candidate genomic intervals. In some instances, the plurality of candidate genomic intervals may comprise at most 100,000, at most 90,000, at most 80,000, at most 70,000, at most 60,000, at most 50,000, at most 40,000, at most 30,000, at most 20,000, at most 10,000, at most 5,000, at most 1,000, at most 900, at most 800, at most 700, at most 600, at most 500, at most 400, at most 300, at most 200, at most 100, at most 90, at most 80, at most 70, at most 60, at most 50, at most 40, at most 30, at most 20, or at most 10 candidate genomic intervals. Any of the lower and upper values described in this paragraph may be combined to form a range included within the present disclosure, for example, in some instances the plurality of candidate genomic intervals may range from about 50 to about 5,000 candidate genomic intervals. Those of skill in the art will recognize that the plurality of candidate genomic intervals may comprise any value within this range, e.g., about 52,500 candidate genomic intervals.

In some instances, the plurality of candidate genomic intervals may comprise between about 1,000 and about 10,000 candidate genomic intervals. In some instances, the plurality of candidate genomic intervals may comprise at least 1,000, at least 2,000, at least 3,000, at least 4,000, at least 5,000, at least 6,000, at least 7,000, at least 8,000, at least 9,000, or at least 10,000 candidate genomic intervals. In some instances, the plurality of candidate genomic intervals may comprise at most 10,000, at most 9,000, at most 8,000, at most 7,000, at most 6,000, at most 5,000, at most 4,000, at most 3,000, at most 2,000, or at most 1,000 candidate genomic intervals. Any of the lower and upper values described in this paragraph may be combined to form a range included within the present disclosure, for example, in some instances the plurality of candidate genomic intervals may range from about 1,000 to about 3,000 candidate genomic intervals. Those of skill in the art will recognize that the plurality of candidate genomic intervals may comprise any value within this range, e.g., about 2,432 candidate genomic intervals.

At step 104 in FIG. 1, annotated patient sample data is received, provided, or generated (e.g., by a system configured to perform process 100). In some instances, the annotated patient sample data can comprise at least one of: (i) methylation sequencing data, and (ii) associated clinical annotation data for a plurality of patients. In some embodiments, the annotated patient sequencing data comprises methylation sequencing data for at least a subset of the plurality of candidate genomic intervals. In some embodiments, the associated clinical annotation data comprises one or more factors associated with a disease or tissue of origin (TOO) for patient samples of the plurality of patient samples.

In some instances, the methylation sequencing data can comprise methylation sequencing data (e.g., the genomic locations of methylated cytosine residues) derived from, e.g., a targeted sequencing method, a whole exome sequencing (WES) method, or a whole genome sequencing (WGS) method. In some instances, the methylation sequencing data can comprise data obtained by sequencing nucleic acid molecules that have been subjected to a cytosine conversion reaction. In some instances, the cytosine conversion reaction may comprise a bisulfite conversion reaction or an enzymatic conversion reaction.

In some instances, the methylation sequencing data can comprise, e.g., the genomic locations of methylated cytosine residues in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the clinical annotation data can comprise, for example, patient gender, age, smoking status, disease type, disease stage, sample type, lab protocol for sample collection and processing, lab protocol for performing methylation sequencing, clinical data for treatment outcomes, or any combination thereof.

In some instances, the annotated patient sample data may comprise methylation sequencing data and/or clinical annotation data for a plurality of patients that have been diagnosed with a disease. In some instances, the annotated patient sample data may comprise methylation sequencing data and/or clinical annotation data for a plurality of patients comprising both patients diagnosed with a disease and healthy patients (e.g., patients not diagnosed with the disease). In some instances, the annotated patient sample data may comprise methylation sequencing data and/or clinical annotation data for a plurality of patients comprising patients diagnosed with a first disease, patients diagnosed with a second disease, and healthy patients (e.g., patients not diagnosed with the first disease or the second disease).

In some instances, the disease (e.g., a first disease and/or a second disease) may comprise a cancer or a genetic disorder. In some instances, the disease is cancer, and the cancer is a colorectal cancer (CRC) or lung cancer. In some instances, the first disease is, e.g., colorectal cancer (CRC) and the second disease is, e.g., lung cancer.

In some instances, the plurality of patients (including patients diagnosed with a disease and/or healthy patients) may comprise between about 100 and about 10,000 patients. In some instances, the plurality of patients may comprise at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, or more than 10,000 patients. In some instances, the plurality of patients may comprise at most 10,000, at most 9000, at most 8000, at most 7000, at most 6000, at most 5000, at most 4000, at most 3000, at most 2000, at most 1000, at most 900, at most 800, at most 700, at most 600, at most 500, at most 400, at most 300, at most 200, or at most 100 patients. Any of the lower and upper values described in this paragraph may be combined to form a range included within the present disclosure, for example, in some instances the plurality of patients may range from about 100 to about 800 patients. Those of skill in the art will recognize that the plurality of patients may comprise any number of patients within this range, e.g., about 1,964 patients.

At step 106 in FIG. 1, an aggregated methylation meta score matrix of candidate methylation meta scores is generated. The aggregated methylation meta score matrix can comprise, for example, candidate methylation meta scores that are based on mathematical combinations of weighted methylation scores assigned to each of the plurality of candidate genomic intervals. In some embodiments, the candidate methylation meta scores that are based on combinations of weighted methylation scores assigned to each of the plurality of candidate genomic intervals.

In some instances, generating the aggregated methylation meta score matrix can comprise: generating a first matrix, where a given cell in the first matrix comprises a methylation score calculated for a corresponding candidate genomic interval based on methylation sequencing data for a corresponding patient sample; generating a second matrix, where a given cell in the second matrix comprises a weighted methylation score assigned to a given candidate genomic interval of the plurality of candidate genomic intervals that indicates how informative the given candidate genomic interval is for disease detection and/or prediction of disease tissue of origin (TOO) in at least a subset of the plurality of patient samples; and combining the first matrix and the second matrix (e.g., by multiplying the first matrix and the second matrix) to generate the aggregated methylation meta score matrix. In some instances, the aggregated methylation meta score matrix is based on the annotated genomic interval data and the annotated patient sample data.

In some instances, the first matrix can comprise a number of rows equal to a total number of patient samples in the plurality of patient samples and a number of columns equal to a total number of candidate genomic intervals in the plurality of candidate genomic intervals.

In some instances, the second matrix can comprise a number of rows equal to a total number of candidate genomic intervals in the plurality of candidate genomic intervals and a number of columns equal to a user-defined number of candidate methylation meta scores.

In some instances, the aggregated methylation meta score matrix can comprise a number of rows equal to the total number of candidate genomic intervals in the plurality of candidate genomic intervals and a number of columns equal to a user-defined number of candidate methylation meta scores. In some instances, a given cell in the aggregated methylation meta score matrix may comprise a candidate methylation meta score based on a mathematical combination of the weighted methylation scores in the second matrix.

In some instances, the methylation score may comprise a percent fully methylated score or a percent fully unmethylated score for a given candidate genomic interval (e.g., a percentage of the available CpG sites within a candidate genomic interval that are methylated, or a percentage of the available CpG sites within a candidate genomic interval that are unmethylated). In some instances, the methylation score may comprise a Cluster Consensus Methylation Fraction (CCMF) score (i.e., the fraction or percentage of fully methylated fragments (e.g., in regions that are hypermethylated in cancer)) or a Cluster Consensus Unmethylated Fraction (CCUF) score (i.e., the faction or percentage of unmethylated fragments (e.g., in regions that are hypomethylated in cancer)).

In some instances, a high value for the weighted methylation score assigned to each candidate genomic interval indicates that the candidate genomic interval is informative for disease detection and/or prediction of disease tissue of origin (TOO). In some instances, a low value for the weighted methylation score assigned to each candidate genomic interval indicates that the candidate genomic interval is not informative for disease detection and/or prediction of disease tissue of origin (TOO). In some instances, the weighted methylation score may be determined, e.g., based on the average ratio of signal-to-noise for the methylation score in cancer vs. non-cancer samples (using either in cancer tissue vs. non-cancer tissue samples, or cancer plasma vs. non-cancer plasma samples), or by determining Area Under the Curve (AUC) or some other metric for predicting cancer vs. non-cancer.

In some instances, the weighted methylation score assigned to each candidate genomic interval of the plurality of candidate genomic intervals is calculated based on a comparison of a methylation metric for each candidate genomic interval for a first subset of the plurality of patient samples to that for each candidate genomic interval for a second subset of the plurality of patient samples. In some instances, the methylation metric can comprise, for example, a CCMF signal-to-noise ratio calculated for each candidate genomic interval for the first and second subsets of the plurality of patient samples (e.g., a ratio of aggregate fully methylated sequence reads in one group (e.g., cancer tissue samples) versus another group (e.g., healthy plasma samples); in some instances, the ratio may be divided by, e.g., the total number of sequence reads; in some instances, the ratio may be modified by, e.g., adding a pseudo-count of +1 for numerator and/or denominator). In some instances, the first subset of the plurality of patient samples may comprise, for example, patient samples from patients diagnosed with a first disease. In some instances, the second subset of the plurality of patient samples may comprise, for example, control samples from patients diagnosed with the first disease. In some instances, the second subset of the plurality of patient samples may comprise, for example, patient samples from patients diagnosed with a second disease.

In some instances, the method (e.g., process 100) may further comprise selecting a subset of the plurality of candidate genomic intervals based on the weighted methylation score assigned to each candidate genomic interval, and modifying the second matrix to reduce the number of rows accordingly.

At step 108 in FIG. 1, the aggregated methylation meta score matrix and the associated clinical annotation data for at least the subset of the plurality of patient samples are input as training data for training a machine learning model.

Examples of suitable machine learning models include, but are not limited to, neural networks (e.g., convolutional neural networks (CNNs)), linear regression models, logistic regression (logit) models, support vector machine (SVM) models, random forest models, or XGBoost models. In some instances, training the machine learning model may comprise using a cross-validation training procedure in which the training data is divided into two parts (a training data set for training the model, and a test data set for testing model performance). The cycle of training the model on the training data set and then validating the model on the test data is repeated until a desired level of model performance is achieved. Examples of suitable cross-validation methods include, but are not limited to, hold-out, K-folds, leave-one-out, leave-p-out, stratified K-folds, repeated K-folds, and nested K-folds cross-validation (see, e.g., Bradshaw et al. (2023), “A Guide to Cross-Validation for Artificial Intelligence in Medical Imaging”, Radiology: Artificial Intelligence 2023; 5(4):e220232; Yates et al. (2023), “Cross Validation for Model Selection: A Review with Examples From Ecology, Ecological Monographs 2023; 93:e1557).

At step 110 in FIG. 1, the machine learning model is trained using the training data to identify an optimal methylation meta score for disease detection, prediction of disease tissue of origin (TOO) for at least one disease type, and/or prediction of a tumor fraction of a sample from a subject based on methylation sequencing data.

In some instances, the method (e.g., process 100) may further comprise providing methylation sequencing data derived from a sample from a subject as input to the trained machine learning model to detect a presence of a disease and/or predict a disease TOO in the subject.

In some instances, the method (e.g., process 100) may further comprise using the trained machine learning model and/or the identified optimal methylation meta score to detect disease in a subject based on methylation sequencing data derived from a sample from the subject.

In some instances, the method (e.g., process 100) may further comprise using the trained machine learning model and/or the identified optimal methylation meta score to predict a disease tissue of origin (TOO) in a subject based on methylation sequencing data derived from a sample from the subject.

In some instances, the method (e.g., process 100) may further comprise using the identified optimal methylation meta score to select a disease treatment for a subject based on methylation sequencing data derived from a sample from the subject.

In some instances, the method (e.g., process 100) may further comprise using the identified optimal methylation meta score to predict a disease treatment outcome for a subject based on methylation sequencing data derived from a sample from the subject.

In some instances, the method (e.g., process 100) may further comprise using the identified optimal methylation meta score to identify a subject for inclusion in a clinical trial for a disease treatment based on methylation sequencing data derived from a sample from the subject.

As noted elsewhere above, in some instances, the disease may be a cancer or a genetic disorder. In some instances, the disease may be a cancer and the method may further comprise selecting an anti-cancer therapy. In some instances, the anti-cancer therapy may comprise chemotherapy, radiation therapy, immunotherapy, a targeted therapy, an neoantigen-based therapy, or surgery.

In some instances, the plurality of patient samples and/or the sample from a subject may comprise tissue biopsy samples, liquid biopsy samples, or a combination thereof. In some instances, the plurality of patient samples and/or the sample from a subject may comprise liquid biopsy samples, and the liquid biopsy samples may comprise blood, plasma, cerebrospinal fluid, sputum, stool, urine, or saliva samples.

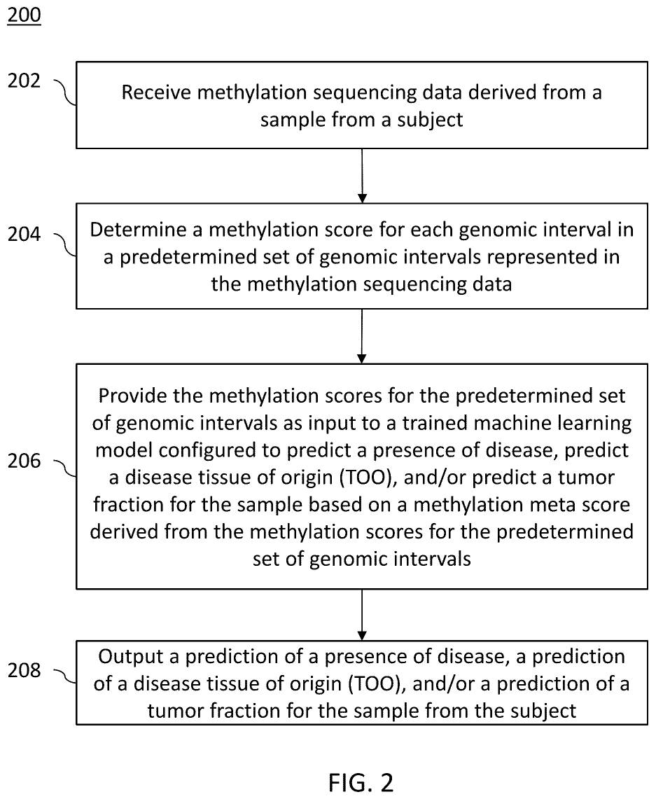

FIG. 2 provides a non-limiting example of a flowchart for a process 200 for predicting a presence of disease and/or a disease tissue of origin for a subject. Process 200 can be, for example, a computer-implemented method, and can performed, for example, using one or more electronic devices implementing a software platform. In some examples, process 200 is performed using a client-server system, and the blocks of process 200 are divided up in any manner between the server and a client device. In other examples, the blocks of process 200 are divided up between the server and multiple client devices. Thus, while portions of process 200 are described herein as being performed by particular devices of a client-server system, it will be appreciated that process 200 is not so limited. In other examples, process 200 is performed using only a client device or only multiple client devices. In process 200, some blocks are, optionally, combined, the order of some blocks is, optionally, changed, and some blocks are, optionally, omitted. In some examples, additional steps may be performed in combination with the process 200. Accordingly, the operations as illustrated (and described in greater detail below) are exemplary by nature and, as such, should not be viewed as limiting.

At step 202 in FIG. 2, methylation sequencing data derived from a sample from a subject is received (e.g., by one or more processors of a system configured to perform process 200).

In some instances, the methylation sequencing data may be generated from one or more sequencing reads associated with the sample. As noted above with respect to FIG. 1, the methylation sequencing data can comprise data (e.g., the genomic locations of methylated cytosine residues) derived from, e.g., a targeted sequencing method, a whole exome sequencing (WES) method, or a whole genome sequencing (WGS) method. In some instances, the methylation sequencing data can comprise data obtained by sequencing nucleic acid molecules that have been subjected to a cytosine conversion reaction. In some instances, the cytosine conversion reaction may comprise a bisulfite conversion reaction or an enzymatic conversion reaction.

In some instances, the sample from the subject may be a tissue biopsy sample or a liquid biopsy sample. In some instances, the sample from the subject may be a liquid biopsy sample, and the liquid biopsy sample may be a blood, plasma, cerebrospinal fluid, sputum, stool, urine, or saliva sample.

At step 204 in FIG. 2, a methylation score is determined for each genomic interval in a predetermined set of genomic intervals represented in the methylation sequencing data.

In some instances, the methylation score can comprise, for example, a methylation fraction score (e.g., a percentage of the available CpG sites within a candidate genomic interval that are methylated), a Cluster Consensus Methylation Fraction (CCMF) score (i.e., the fraction or percentage of fully methylated fragments (e.g., in regions that are hypermethylated in cancer))), an unmethylated fraction score (a percentage of the available CpG sites within a candidate genomic interval that are unmethylated), or a Cluster Consensus Unmethylated Fraction (CCUF) score (i.e., the faction or percentage of unmethylated fragments (e.g., in regions that are hypomethylated in cancer)).

In some instances, the predetermined set of genomic intervals may be identified based on a comparison of the methylation score calculated for each genomic interval represented in the methylation sequencing data to a methylation score threshold. In some instances, the predetermined set of genomic intervals may comprise all genomic intervals for which the calculated methylation score is greater than or equal to the methylation score threshold. In some instances, the methylation score threshold may be disease-specific. In some instances, the methylation score threshold may be disease-independent (e.g., disease agnostic). In some instances, the methylation score threshold may be determined, e.g., based on a collection of training samples and prediction performance targets (e.g., 90, 95, or 99% specificity) and the threshold is set to achieve the desired performance target. For example, setting a 90% specificity target would indicate that 10% of samples known to be disease negative are called as disease positive.

In some instances, the predetermined set of genomic intervals may comprise between about 100 and about 100,000 genomic intervals. In some instances, the predetermined set of genomic intervals may comprise at least 100, at least 200, at least 300, at least 400, at least 500, at least 600, at least 700, at least 800, at least 900, at least 1,000, at least 5,000, at least 10,000, at least 20,000, at least 30,000, at least 40,000, at least 50,000, at least 60,000, at least 70,000, at least 80,000, at least 90,000, at least 100,000, or more than 100,000 genomic intervals. In some instances, the predetermined set of genomic intervals may comprise at most 100,000, at most 90,000, at most 80,000, at most 70,000, at most 60,000, at most 50,000, at most 40,000, at most 30,000, at most 20,000, at most 10,000, at most 5,000, at most 1,000, at most 900, at most 800, at most 700, at most 600, at most 500, at most 400, at most 300, at most 200, or at most 100 genomic intervals. Any of the lower and upper values described in this paragraph may be combined to form a range included within the present disclosure, for example, in some instances the predetermined set of genomic intervals may range from about 1,000 to about 50,000 genomic intervals. Those of skill in the art will recognize that the predetermined set of genomic intervals may comprise any value within this range, e.g., about 12,455 genomic intervals.

At step 206 in FIG. 2, the methylation scores for the predetermined set of genomic intervals are provided as input to a trained machine learning model configured to predict a presence of disease, predict a disease tissue of origin (TOO), and/or predict a tumor fraction for the sample based on a methylation meta score derived from the methylation scores for the predetermined set of genomic intervals.

In some instances, the methylation meta score can comprise an algebraic combination of the methylation scores determined for the predetermined set of genomic intervals and/or tree-based models to determine methylation scores for specific genomic intervals and/or a number of sequence reads that map to each genomic interval of the predetermined set of genomic intervals.

In some instances, as discussed with respect to FIG. 1 above, the machine learning model architecture may be a logistic regression (logit) model or a support vector machine (SVM) model.

In some instances, as discussed with respect to FIG. 1 above, the machine learning model may be trained on a training date set comprising methylation scores determined for a plurality of genomic intervals represented in methylation sequencing data for a first cohort of subjects diagnosed with a first disease.

In some instances, the training data set may further comprise methylation scores determined for the plurality of genomic intervals represented in methylation sequencing data for non-diseased control samples from the first cohort of subjects. In some instances, the non-diseased control samples may be of a different sample type than the diseased samples used to generate the methylation sequencing data for the first cohort of subjects.

In some instances, the training data may further comprise methylation scores determined for the plurality of genomic intervals represented in methylation sequencing data for a second cohort of subjects diagnosed with a second disease.

In some instances, the training data set may further comprise methylation scores determined for the plurality of genomic intervals represented in methylation sequencing data for non-diseased control samples from the second cohort of subjects. In some instances, the non-diseased control samples are of a different sample type than the diseased samples used to generate the methylation sequencing data for the second cohort of subjects.

In some instances, the methylation sequencing data for the second cohort of subjects can be derived from a different sample type than that for the first cohort of subjects.

At step 208 in FIG. 2, a prediction of a presence of disease, a prediction of a disease tissue of origin (TOO), and/or a prediction of a tumor fraction for the sample from the subject is output by the trained machine learning model.

In some instances, the trained machine learning model is configured to predict a presence of the first disease, a presence of the second disease, a disease TOO for the first disease, and/or a disease TOO for the second disease based on a methylation meta score derived from the methylation scores for the predetermined set of genomic intervals.

In some instances, the disease is a cancer or a genetic disorder. In some instances, the disease is cancer, and the cancer is colorectal cancer (CRC) or lung cancer. In some instances, the first disease is colorectal cancer (CRC) and the second disease is lung cancer.

In some instances, the trained machine learning model may be configured to output the methylation meta score, and the method (e.g., process 200) may further comprise using the methylation meta score to select a disease treatment for the subject.

In some instances, the trained machine learning model may be configured to output the methylation meta score, and the method (e.g., process 200) may further comprise using the methylation meta score to predict a disease treatment outcome for the subject.

In some instances, the trained machine learning model may be configured to output the methylation meta score, and the method (e.g., process 200) may further comprise using the methylation meta score to identify the subject for inclusion in a clinical trial for a disease treatment.

In some instance, the methods comprise detection of disease and/or prediction of disease tissue of origin (TOO), comprising: generating, using one or more processors, annotated genomic interval data comprising at least one of: (i) genomic coordinates, and (ii) genomic annotation data associated with candidate genomic intervals of a plurality of candidate genomic intervals; receiving, at the one or more processors, annotated patient sample data comprising (i) methylation sequencing data for at least a subset of the plurality of candidate genomic intervals, and (ii) associated clinical annotation data for a plurality of patient samples, wherein the associated clinical annotation data comprises one or more factors associated with a disease or tissue of origin (TOO) for patient samples of the plurality of patient samples; generating, using the one or more processors, an aggregated methylation meta score matrix comprising candidate methylation meta scores, wherein aggregated methylation meta score matrix is based on the annotated genomic interval data and the annotated patient sample data; inputting, using the one or more processors, (i) the aggregated methylation meta score matrix, and (ii) the associated clinical annotation data for at least a subset of the plurality of patient samples as training data for training a machine learning model; and training, using the one or more processors, the machine learning model using the training data to identify an optimal methylation meta score for disease detection and/or prediction of disease tissue of origin (TOO) for at least one disease type based on methylation sequencing data.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining methylation meta scores may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for determining methylation meta scores may be used to select a subject (e.g., a patient) for a clinical trial based on the methylation meta score value. In some instances, patient selection for clinical trials based on, e.g., a methylation meta score, may accelerate the development of targeted therapies and improve the healthcare outcomes for treatment decisions.

In some instances, the disclosed methods for determining methylation meta scores may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods for determining methylation meta scores may be used in treating a disease (e.g., a cancer) in a subject. For example, in response to determining a methylation meta score for the subject using any of the methods disclosed herein, an effective amount of an anti-cancer therapy or anti-cancer treatment may be administered to the subject.

In some instances, the disclosed methods for determining methylation meta scores may be used for monitoring disease progression or recurrence (e.g., cancer or tumor progression or recurrence) in a subject. For example, in some instances, the methods may be used to determine a methylation meta score in a first sample obtained from the subject at a first time point, and used to determine a methylation meta score in a second sample obtained from the subject at a second time point, where comparison of the first determination of the methylation meta score and the second determination of the methylation meta score allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determined methylation meta score.

In some instances, the value of a methylation meta score determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining methylation meta scores may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining methylation meta scores as part of a genomic profiling process (or inclusion of the output from the disclosed methods for determining methylation meta scores as part of the genomic profile of the subject) can improve the validity of, e.g., disease detection calls and treatment decisions, made on the basis of the genomic profile by, for example, independently confirming the presence of a disease (e.g., a cancer) in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (NMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AMIL), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sezary syndrome, Waldenstrom macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche/454 Genome Sequencer (GS) FLX System, Illumina/Solexa Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS platform. In some instances, sequencing may comprise Illumina MiSeqÔ sequencing. In some instances, sequencing may comprise Illumina HiSeq® sequencing. In some instances, sequencing may comprise Illumina NovaSeq® sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. CàT in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite- or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27(11):1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 March 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11):1571-1572), TARGOMICS (Garinet, et al. (2017), “Calling Chromosome Alterations, DNA Methylation Statuses, and Mutations in Tumors by Simple Targeted Next-Generation Sequencing—A Solution for Transferring Integrated Pangenomic Studies into Routine Practice?”, J. Molecular Diagnostics 19(5):776-787), Bicycle (Grana, et al. (2018) “Bicycle: A Bioinformatics Pipeline to Analyze Bisulfite Sequencing Data”, Bioinformatics 34(8):1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for determining methylation meta scores based on methylation sequencing data from a sample from a subject. The systems may comprise, e.g., one or more processors, and a memory unit communicatively coupled to the one or more processors and configured to store instructions that, when executed by the one or more processors, cause the system to: generate annotated genomic interval data comprising at least one of: (i) genomic coordinates, and (ii) associated genomic annotation data for a plurality of candidate genomic intervals; provide annotated patient sample data comprising at least one of: (i) methylation sequencing data, and (ii) associated clinical annotation data for a plurality of patient samples; generate an aggregated methylation meta score matrix comprising candidate methylation meta scores; input: (i) the aggregated methylation meta score matrix, and (ii) the associated clinical annotation data for at least the subset of the plurality of patient samples as training data for training a machine learning model; and train the machine learning model using the training data to identify an optimal methylation meta score for disease detection and/or prediction of disease tissue of origin (TOO) for at least one disease type based on methylation sequencing data.

Also described herein are system comprising: one or more processors; and a memory communicatively coupled to the one or more processors and configured to store instructions that, when executed by the one or more processors, cause the system to: receive, at one or more processors, methylation sequencing data derived from a sample from a subject; determine, using the one or more processors, a methylation score for each genomic interval in a predetermined set of genomic intervals represented in the methylation sequencing data; provide, to the one or more processors, the methylation scores for the predetermined set of genomic intervals as input to a trained machine learning model configured to predict a presence of disease and/or a disease tissue of origin (TOO) based on a methylation meta score derived from the methylation scores for the predetermined set of genomic intervals; and output, from the one or more processors, a prediction of a presence of disease and/or a disease tissue of origin (TOO) for the subject.

Also disclosed herein are systems that may comprise, e.g., one or more processors, and a memory unit communicatively coupled to the one or more processors and configured to store instructions that, when executed by the one or more processors, cause the system to: receive methylation sequencing data derived from a sample from a subject; determine a methylation score for each genomic interval in a predetermined set of genomic intervals represented in the methylation sequencing data; provide the methylation scores for the predetermined set of genomic intervals as input to a trained machine learning model configured to predict a presence of disease and/or a disease tissue of origin (TOO) based on a methylation meta score derived from the methylation scores for the predetermined set of genomic intervals; and output a prediction of a presence of disease and/or a disease tissue of origin (TOO) for the subject.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for determining a methylation meta score and/or predicting a presence of disease and/or a disease tissue of origin (TOO) for a subject based on methylation sequencing data for the subject, where the methylation sequencing data is derived from any of a variety of samples as described herein (e.g., a tissue sample, biopsy sample, hematological sample, or liquid biopsy sample derived from the subject).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a methylation meta score may be used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Machine Learning

Any of a variety of machine learning approaches & algorithms (where a machine learning model, as referred to herein, comprises a trained machine learning algorithm) may be used in implementing the disclosed methods. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods.

In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using a threshold or activation function, f, where f may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically-distributed operational systems. In some instances, the disclosed methods may employ, for example, pre-trained ANNs, and the pre-trained ANNs can be fine-tuned according to an additional dataset that is inputted into the pre-trained ANN.

### Computer Systems and Networks

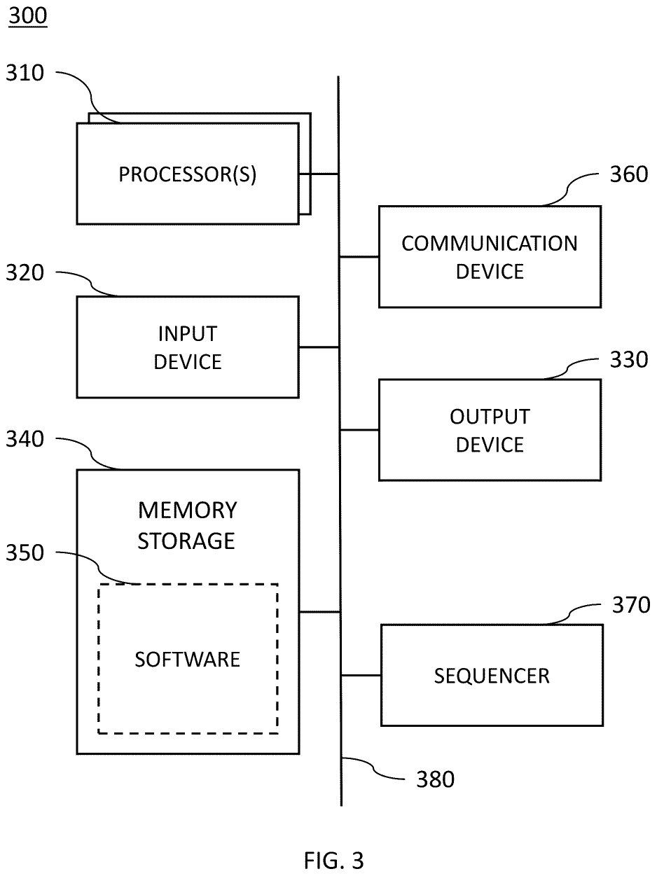

FIG. 3 illustrates an example of a computing device or system in accordance with one embodiment. Device 300 can be a host computer connected to a network. Device 300 can be a client computer or a server. As shown in FIG. 3, device 300 can be any suitable type of microprocessor-based device, such as a personal computer, workstation, server or handheld computing device (portable electronic device) such as a phone or tablet. The device can include, for example, one or more processor(s) 310, input devices 320, output devices 330, memory or storage devices 340, communication devices 360, and nucleic acid sequencers 370. Software 350 residing in memory or storage device 340 may comprise, e.g., an operating system as well as software for executing the methods described herein. Input device 320 and output device 330 can generally correspond to those described herein, and can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage (e.g., an electrical, magnetic or optical memory including a RAM (volatile and non-volatile), cache, hard drive, or removable storage disk). Communication device 360 can include any suitable device capable of transmitting and receiving signals over a network, such as a network interface chip or device. The components of the computer can be connected in any suitable manner, such as via a wired media (e.g., a physical system bus 380, Ethernet connection, or any other wire transfer technology) or wirelessly (e.g., Bluetooth®, Wi-Fi®, or any other wireless technology).

Software module 350, which can be stored as executable instructions in storage 340 and executed by processor(s) 310, can include, for example, an operating system and/or the processes that embody the functionality of the methods of the present disclosure (e.g., as embodied in the devices as described herein).

Software module 350 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 340, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

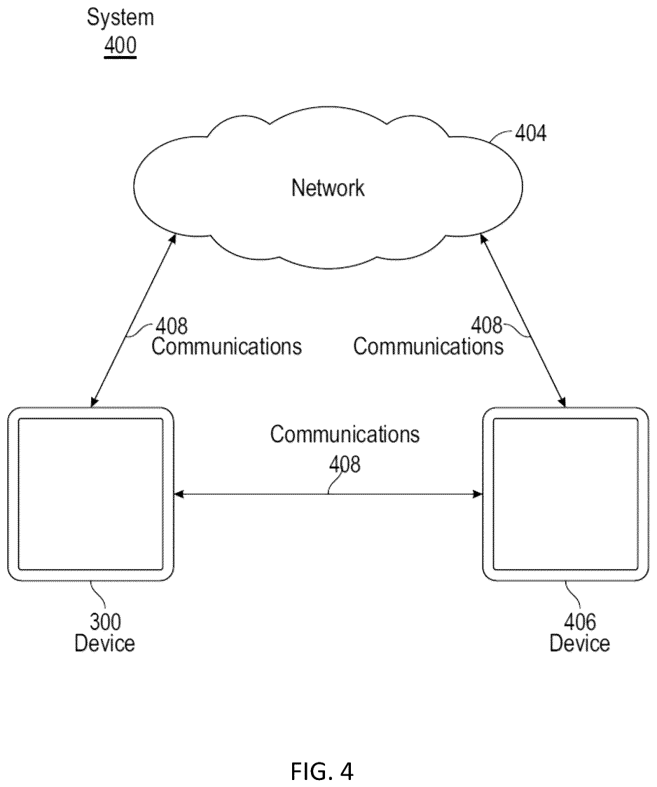

Device 300 may be connected to a network (e.g., network 404, as shown in FIG. 4 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can be implemented using any operating system, e.g., an operating system suitable for operating on the network. Software module 350 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 310.

Device 300 can further include a sequencer 370, which can be any suitable nucleic acid sequencing instrument.

FIG. 4 illustrates an example of a computing system in accordance with one embodiment. In system 400, device 300 (e.g., as described above and illustrated in FIG. 3) is connected to network 404, which is also connected to device 406. In some embodiments, device 406 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, Pacific Biosciences' PacBio® RS system, Ultima Genomics UG 100™ platform, or the Illumina NovaSeq X series platform.

Devices 300 and 406 may communicate, e.g., using suitable communication interfaces via network 404, such as a Local Area Network (LAN), Virtual Private Network (VPN), or the Internet. In some embodiments, network 404 can be, for example, the Internet, an intranet, a virtual private network, a cloud network, a wired network, or a wireless network. Devices 300 and 406 may communicate, in part or in whole, via wireless or hardwired communications, such as Ethernet, IEEE 802.11b wireless, or the like. Additionally, devices 300 and 406 may communicate, e.g., using suitable communication interfaces, via a second network, such as a mobile/cellular network. Communication between devices 300 and 406 may further include or communicate with various servers such as a mail server, mobile server, media server, telephone server, and the like. In some embodiments, Devices 300 and 406 can communicate directly (instead of, or in addition to, communicating via network 404), e.g., via wireless or hardwired communications, such as Ethernet, IEEE 802.11b wireless, or the like. In some embodiments, devices 300 and 406 communicate via communications 408, which can be a direct connection or can occur via a network (e.g., network 404).

One or all of devices 300 and 406 generally include logic (e.g., http web server logic) or are programmed to format data, accessed from local or remote databases or other sources of data and content, for providing and/or receiving information via network 1004 according to various examples described herein.

### EXAMPLES

The following examples are included for illustrative purposes only and are not intended to limit the scope of the present disclosure.

### Example 1—Algorithmic Workflow for Generating Methylation Meta Scores

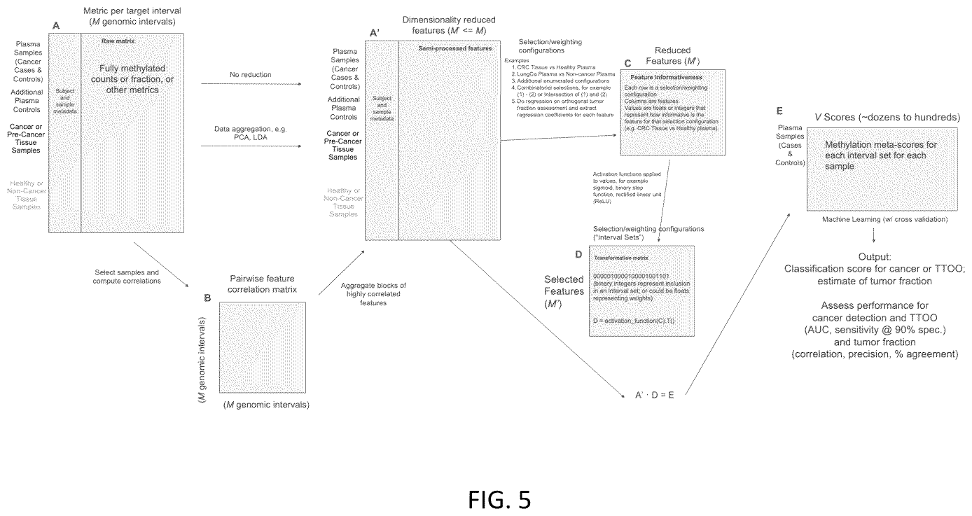

This example describes the algorithmic workflow for generating methylation meta scores, and then using these machine learning-based meta scores to improve cancer prediction and tumor tissue of origin classification based on methylation sequencing data derived from, e.g., plasma samples. The first step of the process is to generate: a) a BED file of candidate genomic intervals to analyze, b) a list of patient samples with links to their methylation sequencing data, and c) clinical metadata for each patient sample (including, e.g., age, gender, smoking status, cancer or disease type, sample type (e.g., plasma, tissue, etc.), and lab protocol for sample preparation, methylation sequencing, etc.). A Matrix A of CCMF values (or other methylation status metrics) is compiled for each patient sample at each candidate genomic interval (Matrix A, FIG. 5). A second Matrix D (i.e., a “Transformation Matrix”) is then created based on groups of patient samples and groups of genomic intervals to be defined subsequently (Matrix D, FIG. 5). Matrix A can be combined with (e.g., multiplied with) Matrix D to generate a matrix of methylation meta scores (Matrix E, FIG. 5). The matrix of methylation meta scores can then be used in training machine learning models, for example, XGBoost with cross validation, to determine the combination of features that is most predictive of cancer and/or of cancer type (i.e., tissue of origin (TOO) or tumor tissue of origin (TTOO)).

To summarize, the matrices depicted in FIG. 5 include:
Matrix A: raw methylation features x samples
Matrix A′: dimensionality reduced features (obtained using, e.g., principal component
analysis (PCA) or linear discriminant analysis (LDA))
Matrix B: pairwise feature correlation matrix to help reduce the features from A to A′.
Matric C: feature importances/weights for a number of selection configurations
Matrix D: transformation matrix; rows correspond to different sample configurations, e.g., CRC vs. non-cancer, lung cancer vs. non-cancer; columns correspond to different methylation features, where the values are feature importance or weight (or included/excluded)
Matrix E: methylation score matrix with dozens of methylation scores each associated with a particular genomic region set (and associated weights), where E=A′ dot D, i.e., the raw scores transformed by the transformation matrix.

The method to derive the transformation matrix (Matrix D, FIG. 5) comprises taking two groups of patient samples, Group A (e.g., lung cancer tissue samples, or stage I/II lung cancer tissue samples, or stage I/II lung adenocarcinoma tissue samples) and Group B (e.g., control plasma samples) and calculating a CCMF signal-to-noise ratio (or other methylation status metric) for these two patient sample groups for each candidate genomic interval. A score is assigned to each genomic interval based on these two groups of patient samples, where a high score denotes that the candidate genomic region is informative and a low score denotes that it is not informative. The importance or weight assigned to each genomic interval may be based on, e.g., the average value of signal to noise ratio of cancer vs. non-cancer samples, or on AUC or another metric of predictiveness of cancer vs. non-cancer. For example if we have N values for cancer and M values for non-cancer samples, one can calculate the average of N/average of M, or one can calculate the AUC of these two arrays.

In a separate process, informative sets of genomic regions are selected and combined (see the conversion of Matrix C to Matrix D in FIG. 5) with weights such that: a) they are maximally informative for Area Under the Curve (AUC) (i.e., a measure of the ability of a binary classifier to distinguish between classes) and sensitivity/specificity, and b) they are non-redundant or minimally redundant (e.g., comprise two genomic intervals that overlap or are located in close proximity). For example, one could just take the top M genomic intervals as ranked by their predictive scores and compute an average score, or one could use a larger subset of candidate genomic intervals and weight each candidate genomic interval by their predictive score. If M is too large (e.g., if one used all of the candidate genomic intervals for prediction of disease and/or TOO), there will be genomic intervals with low predictive scores that can dilute performance. If M is too small (e.g., if one used only the top ranked candidate genomic interval for prediction of disease and/or TOO), then not enough patient samples will include the aberrantly methylated fragments. Different weights can be used. For example, in some instances even weights may be assigned to all genomic intervals having a predictive score that is above a predetermined threshold. In some instances, for example, the threshold may be chosen empirically based on the threshold value that results in the best predictive performance for a training data set (e.g., a percentage of the candidate genomic intervals (such as the top 10%, 5%, 2%, or 1%) or a specified number of candidate genomic intervals (e.g., the top 500, 400, 300, 200, or 100 genomic intervals)). In some instances, weights may be assigned to genomic intervals based on some function of their predictive scores (e.g., a RELU function) with or without a threshold for inclusion.

As noted above, a matrix of methylation meta scores (Matrix E, FIG. 5) can be generated by combining (e.g., multiplying) Matrix A with Matrix D (or by combining (e.g., multiplying) Matrix A′ with Matrix D (see FIG. 5)). The matrix of methylation meta scores (Matrix E, FIG. 5) can then be used in training machine learning models (e.g., using a cross validation loop so that the model is trained and tested on data for different patient samples) to determine the combination of features that is most predictive of cancer and/or of cancer type. One can also develop “Venn Diagram” methylation meta scores by creating a score based on genomic regions that are informative of, e.g., lung cancer, but not informative of, e.g., colorectal cancer, or by creating a score based on genomic regions that are informative of lung cancer AND colorectal cancer.

The algorithmic workflow can be implemented using any of a variety of metrics for methylation status, e.g., CCMF (cluster consensus methylation fraction, i.e., the fraction or percentage of fully methylated fragments (e.g., in regions that are hypermethylated in cancer)), CCUF (cluster consensus unmethylated fraction (i.e., the faction or percentage of unmethylated fragments (in regions that are hypomethylated in cancer)), or other metrics including, but not limited to, K-mer- or Markov Chain-like metrics for determining fragments with aberrant methylation in defined genomic intervals.

A basic implementation of this algorithmic workflow may assume that the predictive power of genomic intervals are independent of each other. If there is much redundancy in the predictive power of the genomic intervals, for example, if they are adjacent or overlapping, the workflow may comprise an additional step performed as part of selecting a subset of genomic intervals to use that comprises merging redundant genomic intervals or selecting one representative (or highest performing) genomic interval. Independent CpG clusters can be selected, for example, by choosing a representative CpG cluster (e.g., a set of N CpG sites within an ˜100 bp region) within a given genomic interval at random or in combination with associated information about the amount of cancer signal it contains.

As described in the example below, we have applied this algorithmic workflow to the detection of colorectal cancer (CRC) and lung cancer with particular thresholds for selecting CpG loci/candidate genomic intervals. We have started exploring this space more generally by varying thresholds, sets of genomic intervals, and tissue sample subsets. A goal of this algorithmic workflow is to enable the exploration and optimization of disease-specific biomarkers through the creation and evaluation of many different methylation meta scores at once.

The algorithmic workflow is illustrated in FIG. 5. One starts with a dataset of methylation sequencing data derived from, e.g., a mix of cancer and control plasma sample data and cancer tissue sample data. Methylation sequencing data can be derived using, e.g., bisulfite sequencing or Enzymatic Methyl (EM) seq techniques, including targeted or whole genome sequencing techniques.

Here, we describe a non-limiting example of implementation of the disclosed workflow for matrix-based CCMF selection and modeling. The goals of the study are to: (i) generate a full-featured matrix of useful CCMF-based methylation data, and (ii) use this matrix to build a generalized set of CCMF-based methylation meta scores, which may then be used to develop cancer detection and tumor tissue of origin (TTOO) classifiers.

This exemplary implementation of the algorithmic workflow includes the steps of:
1) Generation of a matrix of CCMF data for N patient samples across a collection of M CpG cluster candidates (where to start, each candidate genomic interval is assumed to comprise a single CpG cluster in this non-limiting example), where the matrix has dimensions of N×M. A CpG cluster is defined as a short genomic interval containing multiple CpG sites, where the genomic interval starts and ends with CG in the reference sequence. In some instances, a script in the methylation calling tool can generate CCMF-style counts for each patient sample across a collection of CpG clusters identified in a BED reference file. Alternatively, in some instances, candidate genomic intervals may be defined by taking all CpG clusters located in close proximity to each other (e.g., by grouping any CpG clusters within approximately, 150, 200, 250, or 300 bases of each other) and aggregating their data. Since CpG clusters are sparsely distributed across the human genome, this aggregation approach leads to a lower number of distinct genomic intervals. To optimize this proximity aggregation process, one could assess correlation levels as a function of distance (e.g., using an auto-correlation plot).

The number of columns, M, in the matrix of this example is equal to the number of non-redundant CpG clusters (i.e., approximately 15,000 non-redundant CpG clusters). The number of rows, N, in the matrix of this example is equal to the total number of patient samples (i.e., approximately 3,000 patient samples). Methylation sequencing data derived from whole genome bisulfite sequencing (WGBS) was included for cancer tissue samples (e.g., CRC, lung cancer, and others), and plasma control samples. The values included in the matrix can be, for example, methylation counts and/or methylation fractions (and may comprise integer values, fractions ranging in value from 0 to 1, or floating point numbers). In some instances, it is advantageous to keep the matrix small enough to handle without requiring a lot of extra tooling. A matrix comprising data for approximately 3,000 patient samples×15,000 CpG clusters (features) provides about 50 million methylation count and/or methylation fraction values, so the matrix required no more than about 1-2 GB of memory.
2) Generation of a non-negative matrix to transform the raw matrix data into a smaller number, V, of methylation meta scores. This matrix has dimensions of M×V, and includes aggregate scores (e.g., 5-50 aggregate scores) to score each potential methylation meta score-based biomarker on different subsets of samples, e.g., cancer type subsets, healthy subsets, cancer x stage subsets (e.g., lung cancer stage I-II), or subtype subsets (e.g., lung adenocarcinoma). Simple mathematical manipulations may be used for generating candidate methylation meta scores, for example, additional, subtraction, and/or division of CCMF values, including, for example, dividing a cancer CCMF value by a corresponding healthy CCMF value. In some instances, instead of using cancer tissue CCMF values as the “numerator”, one could use values for plasma cancer samples, or values for plasma cancer samples AND cancer tissue samples. Alternatively, one could look specifically for signals that are found in cancer plasma samples but not found in cancer tissue samples.

In some instances, a transformer function providing one-to-one functionality may be used to map methylation meta scores to weights that will be used to compose a transformed matrix. For example, a simple idea could be to use a ReLU function with offset, or a ReLU function combined with a logistic function as a transformer such that a raw M×V matrix is converted to a “transformed” M×V matrix.
3. Calculation of meta-scores for each sample. The resulting matrix has dimensions of N×V and may be generated, e.g., by a matrix multiplication operation such as (N×M)×(M×V)→(N×V).
4) Evaluation of methylation meta scores as predictive biomarkers through the training of a machine learning model using cross-validation. For example, one can start with a simple, highly interpretable model (e.g., a logit or SVM model). A cross-validation training strategy can be employed for modeling in conjunction with a specified set of performance criteria (e.g., an AUC of at least 0.9).

An example process (utilizing 1 to 2 methylation meta-scores) may comprise, for example, aggregating scores for one cancer type in tissue samples (to generate a “foreground” signal); aggregating scores for a collection of control plasma samples (to generate a “background” signal), dividing the foreground signal estimate by the background signal estimate, which constitutes a “feature score”, and using a simple transformer, e.g., an inclusion threshold set so that the features above the threshold are included (values set to “1”) and all others are excluded (values set to “0”).

In some instances, methylation meta scores may be combined to create “Venn” type meta-scores, for example, a “lung minus CRC” methylation meta score, which would require handling 2 to 3 methylation meta scores to generate matrix weights (see, e.g., the selection criteria listed to the left of Matrix C in FIG. 5). As another example, a “lung cancer minus all other cancer types” methylation meta score could be generated, which would result in generation of another matrix of size N2×V2 where V2>V.

In some instances, the number of CpG candidates may be increased to enable the very best CpG clusters to be selected and weighted. For example, a set of 165,000 CpG clusters was generated by considering all 6-mers across the candidate CpG set (while not including other “k” values to limit redundancy).

Inclusion of a larger set of CpG candidates may require strategies to reduce duplicate or “redundant” information in overlapping or nearby CpG clusters. For example one could determine the correlation between all pairs of CpG clusters, and then “prune” CpGs from the M×V matrix by taking the average or maximum CCMF value for a group of correlated CpG clusters in each row of the M×V matrix.

In some instances, machine-driven matrix formation may be utilized, for example, by performing non-negative matrix factorization (NNMF).

In some instances, the process may be repeated to generate and evaluate methylation meta scores based on analysis of a hypomethylation metric (e.g., CCUF) in addition to, or instead of, analysis of a hypermethylation metric (e.g., CCMF).

This algorithmic workflow can also be used to generate and evaluate methylation meta scores as biomarkers for medical conditions or diseases outside of cancer.

### Example 2—Use of Methylation Meta Scores to Detect Cancer and Predict TOO

Many companies are trying to develop methods for detecting cancer based on an analysis of, e.g., cfDNA isolated from plasma or other human specimen types, however, the detection of cancer in asymptomatic individuals, and identification of the type of cancer when detected, is challenging.

One of the advantages of the disclosed methods for generating and evaluating methylation meta scores is that such scores can simultaneously be used for cancer detection and for TTOO. Furthermore, the performance of this approach should be more sensitive for cancer detection than approaches based on any individual methylation metric. This example illustrates the use of methylation meta scores to accurately detect colorectal cancer and lung cancer in asymptomatic individuals, as well as predict tumor of origin.

The disclosed methods are based on determining a methylation score (or metric) for a region set, where the methylation score may be, e.g., CCMF (the count or percentage of fully methylated sequence reads (i.e., deduplicated/unique methylation sequence reads) that align to a given genomic interval), and a region set is defined as a set of genomic intervals for which a given methylation scoring metric is above a predetermined cutoff (e.g., a threshold determined based a specificity threshold as described elsewhere herein). For example, one can calculate the CCMF value at a specified genomic region in a set of samples A and in a set of samples B. One can then calculate a methylation score based on, e.g., the ratio of sum (A)/sum (B) where sum (A) and sum (B) are the sums of CCMF values calculated for each of the samples in sample set A and for each of the samples in sample set B respectively. Alternatively, one could add up a pseudo-count such as (sum (A_fully methylated+1)/sum (A_number of reads+1))/(sum (B_fully methylated+1)/sum (B_number reads+1)). Other methylation score combinations and/or other feature selection criteria can be also used.

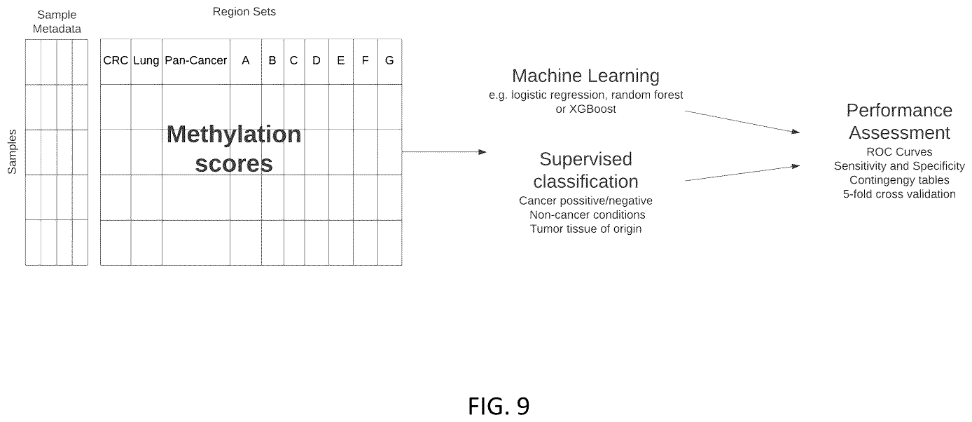

There are a number of differences between the disclosed methods for generating and evaluating methylation meta score-based biomarkers and previous approaches to determining methylation-based biomarkers for cancer detection, including: (a) comparison of methylation data for different sample types (e.g., methylation data for tissue cancer samples and plasma cfDNA data for non-cancer samples), (b) compilation of methylation data for multiple cancer types and healthy sample sets, and (c) generating new genomic region sets based on combinatorial operations performed on existing genomic region sets (e.g., based on the intersection of two genomic region sets, or the subtraction of one genomic region set from another (see the Venn diagram depicted in FIG. 9)).

This example illustrates the use of methylation meta scores to detect trace amounts of CRC and Lung Cancer from plasma samples. Each CCMF score (e.g., a CCMF score for a specified genomic region) may be individually useful for detecting CRC or Lung Cancer, while the matrix of several CCMF scores may be useful to detect CRC or Lung Cancer, or to determine Tissue of Origin.

Tissue of origin in particular is a promising application for this method as there may be significant correlation between CRC methylation scores and lung cancer methylation scores. By creating multiple methylation meta scores through the subtraction and/or intersection of genomic regions sets, and then evaluating these scores through the training of machine learning models, one can accurately predict tumor tissue of origin.

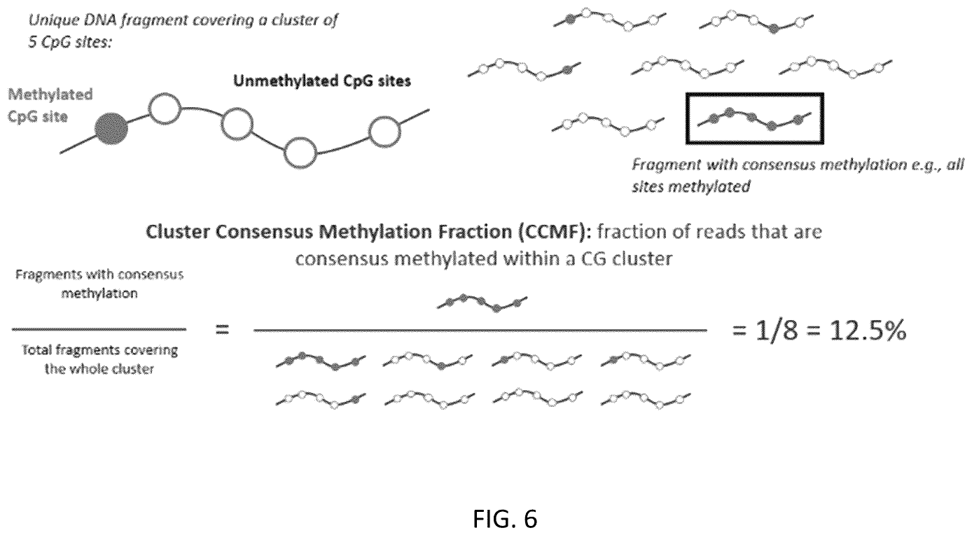

FIG. 6 provides a non-limiting schematic illustration of the calculation of Cluster Consensus Methylation Fraction (CCMF)—one example of a methylation metric that can be used to quantify the methylation status of a region (e.g., a fragment) of genomic DNA. For a given unique DNA fragment comprising one or more CpG sites (e.g., a cluster of five CpG sites, as illustrated in FIG. 6), methylation sequencing can be used to determine how many of the CpG sites comprise methylated cytosine residues in each of a plurality of sequence reads that align to the fragment (e.g., ⅛=12.5%, as illustrated in FIG. 6). The CCMF can then defined as the percentage of sequence reads for which all CpG sites are methylated relative to the total number of sequence reads that align to the fragment. Alternatively, in some instances, a Cluster Consensus Unmethylated Fraction (CCUF) may be determined, where CCUF can be defined as the percentage of sequence reads for which all CpG sites are unmethylated relative to the total number of sequence reads that align to the fragment (e.g., ⅜=37.5% in the example of FIG. 6). Any of a variety of other metrics for quantifying methylation status can also be used.

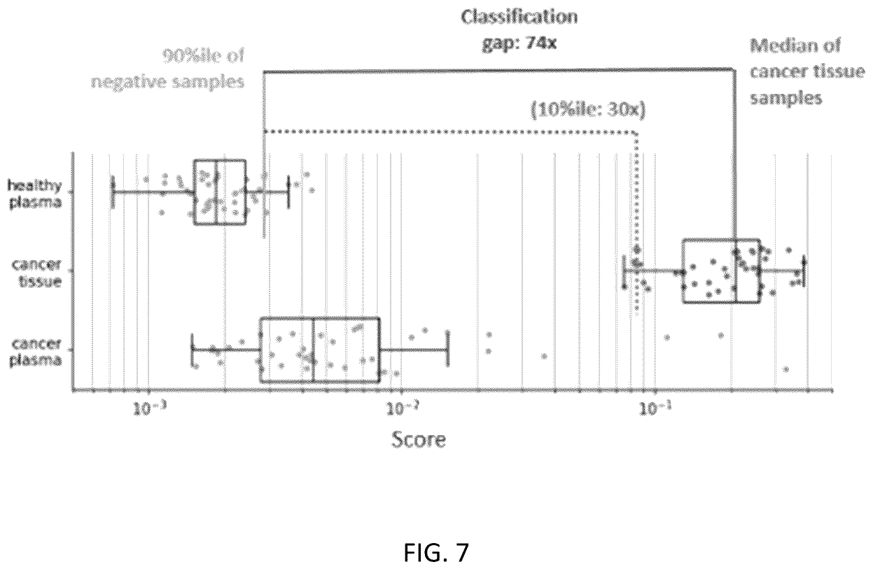

FIG. 7 provides a non-limiting example of data illustrating the challenges of using a methylation score-based assay to discriminate between cancerous and healthy tissue samples. Analytical measurements of methylation status are used to compare samples with aberrant methylation signatures to those without, and are often based on a heuristic for estimating how much dilution of the sample will impact the ability to detect cancer. FIG. 7 provides a box plot of CCUF values computed from methylation sequencing data derived from healthy plasma samples, cancer tissue samples, and cancer plasma samples, as plotted on a log scale. As can be seen in the plot, there was a “classification gap” of approximately 74× between the 90^{th} percentile of CCUF scores for the healthy and the median CCUF score for cancer tissue samples, and a “classification gap” of approximately 30× between the 90^{th} percentile of CCUF scores for the healthy and the 10^{th} percentile of the CCUF scores for the cancer tissue samples. The “classification gap” between the CCUF scores for the healthy samples and those for cancer plasma samples was significantly smaller. Scores that behave linearly when samples are diluted are best suited for discrimination between cancer samples and healthy samples comprising these relatively small classification gaps. The methylation meta scores generated and evaluated using the methods disclosed herein may provide improved detection sensitivity and better performance in prediction tissue of origin.

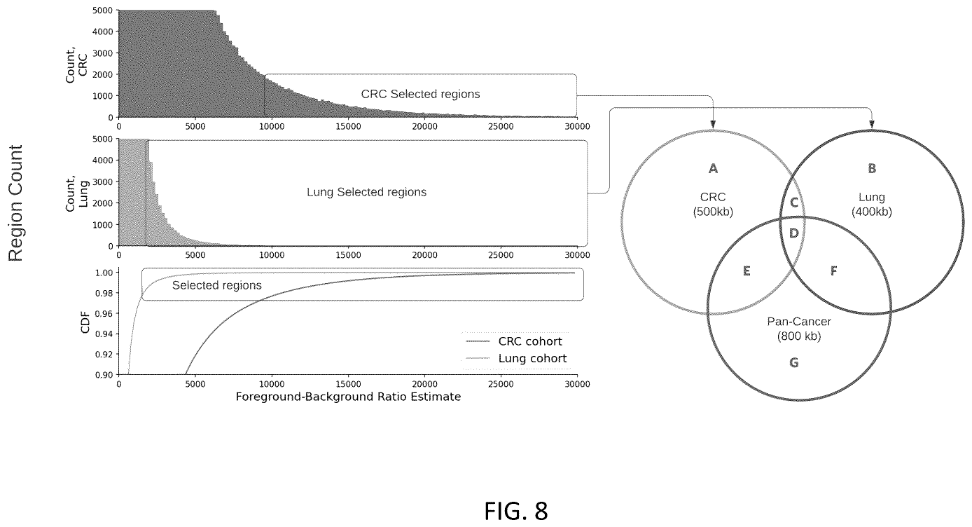

FIG. 8 provides a non-limiting example of methylation data for colorectal cancer (CRC) and lung cancer patients, and illustrates the selection of genomic intervals for generating methylation meta scores for the detection of CRC and lung cancer that differentiate between these cancer types. The upper left panel provides a plot of the number of genomic regions that exhibit a specified methylation foreground-to-background ratio (as determined from sequence reads derived from methylation sequencing of CRC samples that align to each genomic region) as a function of estimated methylation foreground-to-background ratio. The outlined portion of the plot indicates the genomic regions (approximately 500 kb of genomic DNA in total) that were selected for use in generating and evaluating methylation meta scores. The middle left panel provides a plot of the number of genomic regions that exhibit a specified methylation foreground-to-background ratio (as determined from sequence reads derived from methylation sequencing of lung cancer samples that align to each genomic region) as a function of estimated methylation foreground-to-background ratio. Again, the outlined portion of the plot indicates the genomic regions (approximately 400 kb of genomic DNA in total) that were selected for use in generating and evaluating methylation meta scores. The lower left panel provides a plot of the cumulative distribution function (CDF) (i.e., a running sum of the values shown in the plots above, which indicates the number of genomic regions for which the number of sequence reads is less than the foreground-background ratio value) versus methylation foreground-to-background ratio estimates for samples from the CRC (orange) and lung cancer (teal) cohorts. The outlined portion of the plot indicates the range of estimated methylation foreground-to-background ratios for the selected genomic intervals. The right-hand panel in FIG. 8 provides a Venn diagram illustrating the overlap between the selected genomic regions for CRC (approximately 500 kb in total), the selected genomic regions for lung cancer (approximately 400 kb), and genomic regions that were selected for pan-cancer detection (approximately 800 kb in total). Region D represents the genomic regions that are predictive for CRC, lung cancer, and pan-cancer detection. Region C of the Venn diagram represents genomic regions that are predictive for both CRC and lung cancer, and not part of region D. Region E represents genomic regions that are predictive for both CRC and for pan-cancer detection, and not region D. Region F represents genomic regions that are predictive for both lung cancer and pan-cancer detection, and not region D. Region A represents genomic regions that are predictive for CRC and not lung cancer or pan-cancer. Region B represents genomic regions that are predictive for lung cancer and not CRC or pan-cancer. Region C represents genomic regions that are predictive for pan-cancer and not CRC or lung cancer.

FIG. 9 provides a non-limiting schematic illustration of the training data (e.g., Matrix E data (comprising columns A-G corresponding to the sections of the Venn diagram shown in FIG. 8) and sample metadata) used to train a machine learning model and identify an optimal methylation meta score for cancer prediction, and the methods used to assess prediction performance. Training data may comprise both sample metadata (e.g., clinical annotation data, e.g., patient gender, age, smoking status, disease type, disease stage, sample type, lab protocol for sample collection and processing, lab protocol for performing methylation sequencing, clinical data for treatment outcomes, or any combination thereof) for a plurality of patient samples, and methylation meta scores computed for each of a plurality of genomic region sets for each of the patient samples. The data is used to train a machine learning model (e.g., a logistic regression model, a support vector machine (SVM) model, a random forest model, or an XGBoost model) using a supervised learning technique (e.g., using labeled training data comprising cancer positive sample data, cancer negative (healthy) sample data, non-cancer disease sample data, tumor tissue of origin sample data, etc.) and cross-validation (e.g., 5-fold cross-validation). The training process allows selection of an optimal methylation meta score for detecting disease (e.g., cancer) and/or predicting tissue of origin (e.g., tumor tissue of origin). The performance of the trained model for detecting disease and/or predicting tumor tissue of origin can be evaluated using any of a variety of techniques known to those of skill in the art including, but not limited to, receiver operating characteristic (ROC) curves, sensitivity and specificity determinations, contingency tables, etc.

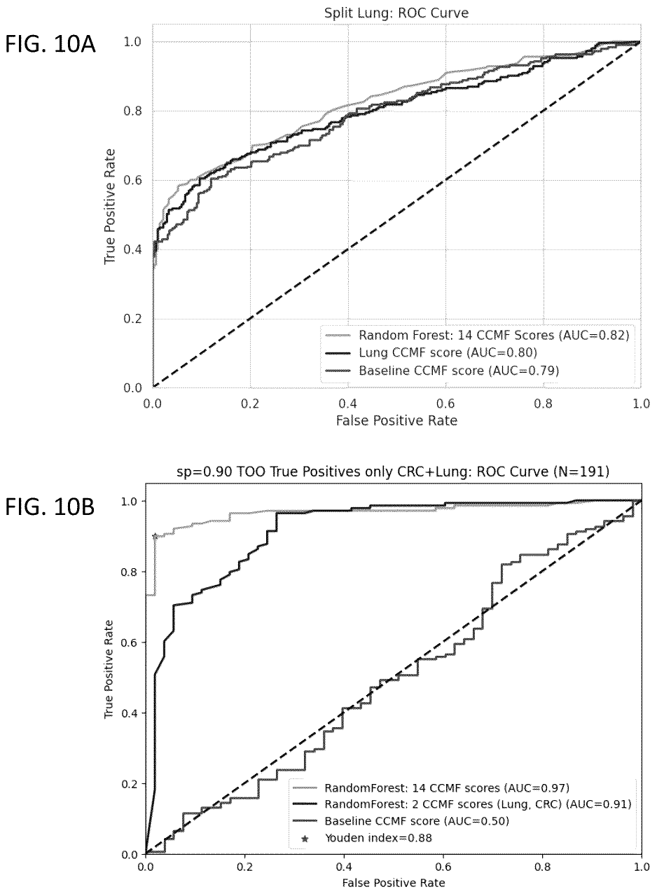

FIG. 10 provides two non-limiting example of receiver operator characteristic (ROC) curves for methylation meta score-based machine learning models used for lung cancer detection (FIG. 10A) and tumor tissue of origin classification (FIG. 10B). True positive rate is plotted as a function of false positive rate for the results of lung cancer detection (FIG. 10A), obtained using three different machine learning-based classifiers. True positive rate is plotted as a function of false positive rate for the results of tumor tissue of origin (TOO) prediction (FIG. 10B; with specificity set to 0.90 for TOO true positives) in 191 patient samples (CRC and lung cancer samples) obtained using three different machine learning-based classifiers. Baseline data for TOO prediction based on a simple CCMF score is also shown. The area-under-the-curve (AUC) metrics for the plots obtained using the different models are shown in the inset. A Jouden Index value of 0.88 was obtained for tumor tissue of origin classification (FIG. 10B).

### EXEMPLARY IMPLEMENTATIONS

Exemplary implementations of the methods and systems described herein include:
1. A method for detection of disease and/or prediction of disease tissue of origin (TOO), the method comprising:
   generating, using one or more processors, annotated genomic interval data comprising at least one of: (i) genomic coordinates, and (ii) associated genomic annotation data for a plurality of candidate genomic intervals;
   providing, using the one or more processors, annotated patient sample data comprising at least one of: (i) methylation sequencing data, and (ii) associated clinical annotation data for a plurality of patient samples;
   generating, using the one or more processors, an aggregated methylation meta score matrix comprising candidate methylation meta scores;
   inputting, using the one or more processors, (i) the aggregated methylation meta score matrix, and (ii) the associated clinical annotation data for at least a subset of the plurality of patient samples as training data for training a machine learning model; and
   training, using the one or more processors, the machine learning model using the training data to identify an optimal methylation meta score for disease detection and/or prediction of disease tissue of origin (TOO) for at least one disease type based on methylation sequencing data.
2. The method of clause 1, further comprising providing methylation sequencing data derived from a sample from a subject as input to the trained machine learning model to detect a presence of a disease and/or predict a disease TOO in the subject.
3. The method of clause 1 or clause 2, wherein the annotated genomic interval data comprises a Browser Extensible Data (BED) file.
4. The method of any one of clauses 1 to 3, wherein the genomic annotation data comprises gene or partial gene sequence identification, promoter sequence coordinates, regulatory sequence coordinates, enhancer sequence coordinates, transcription start sites, open chromatin regions, known CpG cluster locations, known aberrant methylation pattern locations, or any combination thereof for each candidate genomic interval of the plurality.
5. The method of any one of clauses 1 to 4, wherein the candidate methylation meta scores in the aggregated methylation meta score matrix are based on mathematical combinations of weighted methylation scores assigned to each of the plurality of candidate genomic intervals.
6. The method of any one of clauses 1 to 5, wherein generating the aggregated methylation meta score matrix comprises:
   generating, using the one or more processors, a first matrix, wherein a given cell in the first matrix comprises a methylation score calculated for a corresponding candidate genomic interval based on methylation sequencing data for a corresponding patient sample;
   generating, using the one or more processors, a second matrix, wherein a given cell in the second matrix comprises a weighted methylation score assigned to a given candidate genomic interval of the plurality of candidate genomic intervals that indicates how informative the given candidate genomic interval is for disease detection and/or prediction of disease tissue of origin (TOO) in at least a subset of the plurality of patient samples; and
   combining, using the one or more processors, the first matrix and the second matrix to generate the aggregated methylation meta score matrix.
7. The method of clause 6, wherein the first matrix comprises a number of rows equal to a total number of patient samples in the plurality of patient samples and a number of columns equal to a total number of candidate genomic intervals in the plurality of candidate genomic intervals.
8. The method of clause 6 or clause 7, wherein the second matrix comprises a number of rows equal to a total number of candidate genomic intervals in the plurality of candidate genomic intervals and a number of columns equal to a user-defined number of candidate methylation meta scores.
9. The method of any one of clauses 1 to 8, wherein the aggregated methylation meta score matrix comprises a number of rows equal to the total number of candidate genomic intervals in the plurality of candidate genomic intervals and a number of columns equal to a user-defined number of candidate methylation meta scores.
10. The method of one of clauses 5 to 9, wherein a given cell in the aggregated methylation meta score matrix comprises a candidate methylation meta score based on a mathematical combination of the weighted methylation scores in the second matrix.
11. The method of any one of clauses 1 to 10, wherein training the machine learning model comprises using a cross-validation training procedure.
12. The method of any one of clauses 1 to 11, wherein the methylation sequencing data comprises methylation sequencing data derived from a targeted sequencing method, a whole exome sequencing (WES) method, or a whole genome sequencing (WGS) method.
13. The method of any one of clauses 1 to 12, wherein the methylation sequencing data comprises data obtained by sequencing nucleic acid molecules that have been subjected to a cytosine conversion reaction.
14. The method of clause 13, wherein the cytosine conversion reaction comprises a bisulfite conversion reaction or an enzymatic conversion reaction.
15. The method of any one of clauses 1 to 14, wherein the clinical annotation data comprises patient gender, age, smoking status, disease type, disease stage, sample type, lab protocol for sample collection and processing, lab protocol for performing methylation sequencing, clinical data for treatment outcomes, or any combination thereof.
16. The method of any one of clauses 1 to 15, wherein the methylation score comprises a percent fully methylated score or a percent fully unmethylated score.
17. The method of any one of clauses 1 to 16, wherein a high value for the weighted methylation score assigned to each candidate genomic interval indicates that the candidate genomic interval is informative for disease detection and/or prediction of disease tissue of origin (TOO).
18. The method of any one of clauses 1 to 17, wherein a low value for the weighted methylation score assigned to each candidate genomic interval indicates that the candidate genomic interval is not informative for disease detection and/or prediction of disease tissue of origin (TOO).
19. The method of any one of clauses 1 to 18, wherein the weighted methylation score assigned to each candidate genomic interval of the plurality of candidate genomic intervals is calculated based on a comparison of a methylation metric for each candidate genomic interval for a first subset of the plurality of patient samples to that for each candidate genomic interval for a second subset of the plurality of patient samples.
20. The method of clause 19, wherein the methylation metric comprises a CCMF signal-to-noise ratio calculated for each candidate genomic interval for the first and second subsets of the plurality of patient samples.
21. The method of clause 19 or clause 20, wherein the first subset of the plurality of patient samples comprises patient samples from patients diagnosed with a first disease.
22. The method of any one of clauses 19 to 21, wherein the second subset of the plurality of patient samples comprises control samples from patients diagnosed with the first disease.
23. The method of any one of clauses 19 to 21, wherein the second subset of the plurality of patient samples comprises patient samples from patients diagnosed with a second disease.
24. The method of any one of clauses 1 to 23, further comprising selecting a subset of the plurality of candidate genomic intervals based on the weighted methylation score assigned to each candidate genomic interval and modifying the second matrix to reduce the number of rows accordingly.
25. The method of any one of clauses 1 to 24, further comprising using the identified optimal methylation meta score to detect disease in a subject based on methylation sequencing data derived from a sample from the subject.
26. The method of any one of clauses 1 to 25, further comprising using the identified optimal methylation meta score to predict a disease tissue of origin (TOO) in a subject based on methylation sequencing data derived from a sample from the subject.
27. The method of any one of clauses 1 to 26, further comprising using the identified optimal methylation meta score to select a disease treatment for a subject based on methylation sequencing data derived from a sample from the subject.
28. The method of any one of clauses 1 to 27, further comprising using the identified optimal methylation meta score to predict a disease treatment outcome for a subject based on methylation sequencing data derived from a sample from the subject.
29. The method of any one of clauses 1 to 28, further comprising using the identified optimal methylation meta score to identify a subject for inclusion in a clinical trial for a disease treatment based on methylation sequencing data derived from a sample from the subject.
30. The method of any one of clauses 1 to 29, wherein the disease is a cancer or a genetic disorder.
31. The method of clause 30, wherein the disease is cancer, and the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
32. The method of any one of clauses 27 to 31, wherein the disease is a cancer, and the disease treatment comprises an anti-cancer therapy.
33. The method of clause 32, wherein the anti-cancer therapy comprises chemotherapy, radiation therapy, immunotherapy, a targeted therapy, an neoantigen-based therapy, or surgery.
34. The method of any one of clauses 1 to 33, wherein the plurality of patient samples comprises tissue biopsy samples, liquid biopsy samples, or a combination thereof.
35. The method of clause 34, wherein the plurality of patient samples comprises liquid biopsy samples, and the liquid biopsy samples comprise blood, plasma, cerebrospinal fluid, sputum, stool, urine, or saliva samples.
36. A method for detection of disease and/or prediction of disease tissue of origin (TOO), the method comprising: receiving, at one or more processors, methylation sequencing data derived from a sample from a subject;
   determining, using the one or more processors, a methylation score for each genomic interval in a predetermined set of genomic intervals represented in the methylation sequencing data;
   providing, using the one or more processors, the methylation scores for the predetermined set of genomic intervals as input to a trained machine learning model configured to predict a presence of disease and/or a disease tissue of origin (TOO) based on a methylation meta score derived from the methylation scores for the predetermined set of genomic intervals; and
   outputting, using the one or more processors, a prediction of a presence of disease and/or a disease tissue of origin (TOO) for the subject.
37. The method of clause 36, wherein the methylation sequencing data is generated from one or more sequencing reads associated with the sample.
38. The method of clause 36 or clause 37, wherein the methylation score comprises a methylation fraction score, a Cluster Consensus Methylation Fraction (CCMF) score, an unmethylated fraction score, or a Cluster Consensus Unmethylated Fraction (CCUF) score.
39. The method of any one of clauses 36 to 38, wherein the predetermined set of genomic intervals is identified based on a comparison of the methylation score calculated for each genomic interval represented in the methylation sequencing data to a methylation score threshold.
40. The method of clause 39, wherein the predetermined set of genomic intervals comprises all genomic intervals for which the calculated methylation score is greater than or equal to the methylation score threshold.
41. The method of clause 39 or clause 40, wherein the methylation score threshold is disease-specific.
42. The method of clause 39 or clause 40, wherein the methylation score threshold is disease-independent.
43. The method of any one of clauses 36 to 42, wherein the methylation meta score comprises an algebraic combination of the methylation scores determined for the predetermined set of genomic intervals and/or tree-based models to determine methylation scores for specific genomic intervals and/or a number of sequence reads that map to each genomic interval of the predetermined set of genomic intervals.
44. The method of any one of clauses 36 to 43, wherein the machine learning model is trained on a training date set comprising methylation scores determined for a plurality of genomic intervals represented in methylation sequencing data for a first cohort of subjects diagnosed with a first disease.
45. The method of clause 44, wherein the training data set further comprises methylation scores determined for the plurality of genomic intervals represented in methylation sequencing data for non-diseased control samples from the first cohort of subjects.
46. The method of clause 45, wherein the non-diseased control samples are of a different sample type than diseased samples used to generate the methylation sequencing data for the first cohort of subjects.
47. The method of any one of clauses 44 to 46, wherein the training data further comprises methylation scores determined for the plurality of genomic intervals represented in methylation sequencing data for a second cohort of subjects diagnosed with a second disease.
48. The method of clause 47, wherein the training data set further comprises methylation scores determined for the plurality of genomic intervals represented in methylation sequencing data for non-diseased control samples from the second cohort of subjects.
49. The method of clause 48, wherein the non-diseased control samples are of a different sample type than diseased samples used to generate the methylation sequencing data for the second cohort of subjects.
50. The method of any one of clauses 47 to 49, wherein the methylation sequencing data for the second cohort of subjects is derived from a different sample type than that for the first cohort of subjects.
51. The method of any one of clauses 47 to 50, wherein the trained machine learning model is configured to predict a presence of the first disease, a presence of the second disease, a disease TOO for the first disease, and/or a disease TOO for the second disease based on a methylation meta score derived from the methylation scores for the predetermined set of genomic intervals
52. The method of any one of clauses 36 to 51, wherein the disease is cancer or a genetic disorder.
53. The method of any one of clauses 36 to 52, wherein the disease is cancer, and the cancer is colorectal cancer (CRC) or lung cancer.
54. The method of any one of clauses 47 to 53, wherein the first disease is colorectal cancer (CRC) and the second disease is lung cancer.
55. The method of any one of clauses 36 to 54, wherein the trained machine learning model is configured to output the methylation meta score, and wherein the method further comprises using the methylation meta score to select a disease treatment for the subject.
56. The method of any one of clauses 36 to 55, wherein the trained machine learning model is configured to output the methylation meta score, and wherein the method further comprises using the methylation meta score to predict a disease treatment outcome for the subject.
57. The method of any one of clauses 36 to 56, wherein the trained machine learning model is configured to output the methylation meta score, and wherein the method further comprises using the methylation meta score to identify the subject for inclusion in a clinical trial for a disease treatment.
58. The method of any one of clauses 36 to 57, wherein the methylation sequencing data comprises methylation sequencing data derived from a targeted sequencing method, a whole exome sequencing (WES) method, or a whole genome sequencing (WGS) method.
59. The method of any one of clauses 36 to 58, wherein the methylation sequencing data comprises bisulfite sequencing data, enzymatic methyl (EM) sequencing data, or a combination thereof.
60. The method of any one of clauses 36 to 59, wherein the sample from the subject is a tissue biopsy sample or a liquid biopsy sample.
61. The method of clause 60, wherein the sample from the subject is a liquid biopsy sample, and the liquid biopsy sample is a blood, plasma, cerebrospinal fluid, sputum, stool, urine, or saliva sample.
62. A system comprising:
   one or more processors; and
   a memory communicatively coupled to the one or more processors and configured to store instructions that, when executed by the one or more processors, cause the system to perform the method of any one of clauses 1 to 61.
63. A non-transitory computer-readable storage medium storing one or more programs, the one or more programs comprising instructions, which when executed by one or more processors of a system, cause the system to perform the method of any one of clauses 1 to 61.
64. A method for detection of disease, prediction of disease tissue of origin (TOO), and/or calculation of a tumor quantity metric, the method comprising:
   generating, using one or more processors, annotated genomic interval data comprising at least one of: (i) genomic coordinates, and (ii) associated genomic annotation data for a plurality of candidate genomic intervals;
   providing, using the one or more processors, annotated patient sample data comprising at least one of: (i) methylation sequencing data, and (ii) associated clinical annotation data for a plurality of patient samples;
   generating, using the one or more processors, an aggregated methylation meta score matrix comprising candidate methylation meta scores;
   inputting, using the one or more processors, (i) the aggregated methylation meta score matrix, and (ii) the associated clinical annotation data for at least a subset of the plurality of patient samples as training data for training a machine learning model; and
   training, using the one or more processors, the machine learning model using the training data to identify an optimal methylation meta score for disease detection, prediction of disease tissue of origin (TOO) for at least one disease type, and/or calculation of a tumor quantity metric based on methylation sequencing data.
65. The method of clause 64, wherein the tumor quantity metric is a tumor fraction.
66. A method for detection of disease and/or prediction of disease tissue of origin (TOO), the method comprising:
   generating, using one or more processors, annotated genomic interval data comprising at least one of: (i) genomic coordinates, and (ii) genomic annotation data associated with candidate genomic intervals of a plurality of candidate genomic intervals;
   receiving, at the one or more processors, annotated patient sample data comprising (i) methylation sequencing data for at least a subset of the plurality of candidate genomic intervals, and (ii) associated clinical annotation data for a plurality of patient samples, wherein the associated clinical annotation data comprises one or more factors associated with a disease or tissue of origin (TOO) for patient samples of the plurality of patient samples;
   generating, using the one or more processors, an aggregated methylation meta score matrix comprising candidate methylation meta scores, wherein aggregated methylation meta score matrix is based on the annotated genomic interval data and the annotated patient sample data;
   inputting, using the one or more processors, (i) the aggregated methylation meta score matrix, and (ii) the associated clinical annotation data for at least a subset of the plurality of patient samples as training data for training a machine learning model; and
   training, using the one or more processors, the machine learning model using the training data to identify an optimal methylation meta score for disease detection and/or prediction of disease tissue of origin (TOO) for at least one disease type based on methylation sequencing data.
67. The method of clause 66, further comprising providing methylation sequencing data derived from a sample from a subject as input to the trained machine learning model to detect a presence of a disease and/or predict a disease TOO in the subject.
68. The method of clause 66, wherein the genomic annotation data comprises gene or partial gene sequence identification, promoter sequence coordinates, regulatory sequence coordinates, enhancer sequence coordinates, transcription start sites, open chromatin regions, known CpG cluster locations, known aberrant methylation pattern locations, or any combination thereof for each candidate genomic interval of the plurality.
69. The method of clause 66, wherein the candidate methylation meta scores in the aggregated methylation meta score matrix are based on combinations of weighted methylation scores assigned to each of the plurality of candidate genomic intervals.
70. The method of clause 66, wherein generating the aggregated methylation meta score matrix comprises:
   generating, using the one or more processors, a first matrix, wherein a given cell in the first matrix comprises a methylation score calculated for a corresponding candidate genomic interval based on methylation sequencing data for a corresponding patient sample;
   generating, using the one or more processors, a second matrix, wherein a given cell in the second matrix comprises a weighted methylation score assigned to a given candidate genomic interval of the plurality of candidate genomic intervals that indicates how informative the given candidate genomic interval is for disease detection and/or prediction of disease tissue of origin (TOO) in at least a subset of the plurality of patient samples; and
   combining, using the one or more processors, the first matrix and the second matrix to generate the aggregated methylation meta score matrix.
71. The method of clause 70, wherein the first matrix comprises a number of rows equal to a total number of patient samples in the plurality of patient samples and a number of columns equal to a total number of candidate genomic intervals in the plurality of candidate genomic intervals or wherein the second matrix comprises a number of rows equal to a total number of candidate genomic intervals in the plurality of candidate genomic intervals and a number of columns equal to a user-defined number of candidate methylation meta scores.
72. The method of clause 66, wherein the aggregated methylation meta score matrix comprises a number of rows equal to the total number of candidate genomic intervals in the plurality of candidate genomic intervals and a number of columns equal to a user-defined number of candidate methylation meta scores.
73. The method of clause 66, wherein the clinical annotation data comprises patient gender, age, smoking status, disease type, disease stage, sample type, lab protocol for sample collection and processing, lab protocol for performing methylation sequencing, clinical data for treatment outcomes, or any combination thereof.
74. The method of clause 66, wherein the weighted methylation score assigned to each candidate genomic interval of the plurality of candidate genomic intervals is calculated based on a comparison of a methylation metric for each candidate genomic interval for a first subset of the plurality of patient samples to that for each candidate genomic interval for a second subset of the plurality of patient samples.
75. The method of clause 74, wherein the methylation metric comprises a CCMF signal-to-noise ratio calculated for each candidate genomic interval for the first and second subsets of the plurality of patient samples.
76. The method of clause 74, wherein the first subset of the plurality of patient samples comprises patient samples from patients diagnosed with a first disease or wherein the second subset of the plurality of patient samples comprises control samples from patients diagnosed with the first disease or wherein the second subset of the plurality of patient samples comprises patient samples from patients diagnosed with a second disease.
77. The method of clause 66, further comprising selecting a subset of the plurality of candidate genomic intervals based on the weighted methylation score assigned to each candidate genomic interval and modifying the second matrix to reduce the number of rows accordingly.
78. The method of clause 66, further comprising using the identified optimal methylation meta score to detect disease in a subject based on methylation sequencing data derived from a sample from the subject.
79. The method of clause 66, further comprising using the identified optimal methylation meta score (1) to predict a disease tissue of origin (TOO) in a subject based on methylation sequencing data derived from a sample from the subject, (2) to select a disease treatment for a subject based on methylation sequencing data derived from a sample from the subject, (3) to predict a disease treatment outcome for a subject based on methylation sequencing data derived from a sample from the subject, or (4) to identify a subject for inclusion in a clinical trial for a disease treatment based on methylation sequencing data derived from a sample from the subject.
80. A method for detection of disease and/or prediction of disease tissue of origin (TOO), the method comprising:
   receiving, at one or more processors, methylation sequencing data derived from a sample from a subject;
   determining, using the one or more processors, a methylation score for each genomic interval in a predetermined set of genomic intervals represented in the methylation sequencing data;
   providing, to the one or more processors, the methylation scores for the predetermined set of genomic intervals as input to a trained machine learning model configured to predict a presence of disease and/or a disease tissue of origin (TOO) based on a methylation meta score derived from the methylation scores for the predetermined set of genomic intervals; and
   outputting, from the one or more processors, a prediction of a presence of disease and/or a disease tissue of origin (TOO) for the subject.
81. The method of clause 80, wherein the methylation score comprises a methylation fraction score, a Cluster Consensus Methylation Fraction (CCMF) score, an unmethylated fraction score, or a Cluster Consensus Unmethylated Fraction (CCUF) score.
82. The method of clause 80, wherein the predetermined set of genomic intervals is identified based on a comparison of the methylation score calculated for each genomic interval represented in the methylation sequencing data to a methylation score threshold.
83. The method of clause 80, wherein the methylation meta score comprises an algebraic combination of the methylation scores determined for the predetermined set of genomic intervals and/or tree-based models to determine methylation scores for specific genomic intervals and/or a number of sequence reads that map to each genomic interval of the predetermined set of genomic intervals.
84. The method of clause 80, wherein the trained machine learning model is configured to output the methylation meta score, and wherein the method further comprises using the methylation meta score (1) to select a disease treatment for the subject, (2) to predict a disease treatment outcome for the subject, or (3) to identify the subject for inclusion in a clinical trial for a disease treatment.
85. A system comprising:
   one or more processors; and
   a memory communicatively coupled to the one or more processors and configured to store instructions that, when executed by the one or more processors, cause the system to:
      receive, at one or more processors, methylation sequencing data derived from a sample from a subject;
      determine, using the one or more processors, a methylation score for each genomic interval in a predetermined set of genomic intervals represented in the methylation sequencing data;
      provide, to the one or more processors, the methylation scores for the predetermined set of genomic intervals as input to a trained machine learning model configured to predict a presence of disease and/or a disease tissue of origin (TOO) based on a methylation meta score derived from the methylation scores for the predetermined set of genomic intervals; and
      output, from the one or more processors, a prediction of a presence of disease and/or a disease tissue of origin (TOO) for the subject.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method for detection of disease and/or prediction of disease tissue of origin (TOO), the method comprising:
generating, using one or more processors, annotated genomic interval data comprising at least one of: (i) genomic coordinates, and (ii) genomic annotation data associated with candidate genomic intervals of a plurality of candidate genomic intervals;
receiving, at the one or more processors, annotated patient sample data comprising (i) methylation sequencing data for at least a subset of the plurality of candidate genomic intervals, and (ii) associated clinical annotation data for a plurality of patient samples, wherein the associated clinical annotation data comprises one or more factors associated with a disease or tissue of origin (TOO) for patient samples of the plurality of patient samples;
generating, using the one or more processors, an aggregated methylation meta score matrix comprising candidate methylation meta scores, wherein aggregated methylation meta score matrix is based on the annotated genomic interval data and the annotated patient sample data;
inputting, using the one or more processors, (i) the aggregated methylation meta score matrix, and (ii) the associated clinical annotation data for at least a subset of the plurality of patient samples as training data for training a machine learning model; and
training, using the one or more processors, the machine learning model using the training data to identify an optimal methylation meta score for disease detection and/or prediction of disease tissue of origin (TOO) for at least one disease type based on methylation sequencing data.

2. The method of claim 1, further comprising providing methylation sequencing data derived from a sample from a subject as input to the trained machine learning model to detect a presence of a disease and/or predict a disease TOO in the subject.

3. The method of claim 1, wherein the genomic annotation data comprises gene or partial gene sequence identification, promoter sequence coordinates, regulatory sequence coordinates, enhancer sequence coordinates, transcription start sites, open chromatin regions, known CpG cluster locations, known aberrant methylation pattern locations, or any combination thereof for each candidate genomic interval of the plurality.

4. The method of claim 1, wherein the candidate methylation meta scores in the aggregated methylation meta score matrix are based on combinations of weighted methylation scores assigned to each of the plurality of candidate genomic intervals.

5. The method of claim 1, wherein generating the aggregated methylation meta score matrix comprises:
generating, using the one or more processors, a first matrix, wherein a given cell in the first matrix comprises a methylation score calculated for a corresponding candidate genomic interval based on methylation sequencing data for a corresponding patient sample;
generating, using the one or more processors, a second matrix, wherein a given cell in the second matrix comprises a weighted methylation score assigned to a given candidate genomic interval of the plurality of candidate genomic intervals that indicates how informative the given candidate genomic interval is for disease detection and/or prediction of disease tissue of origin (TOO) in at least a subset of the plurality of patient samples; and
combining, using the one or more processors, the first matrix and the second matrix to generate the aggregated methylation meta score matrix.

6. The method of claim 5, wherein the first matrix comprises a number of rows equal to a total number of patient samples in the plurality of patient samples and a number of columns equal to a total number of candidate genomic intervals in the plurality of candidate genomic intervals or wherein the second matrix comprises a number of rows equal to a total number of candidate genomic intervals in the plurality of candidate genomic intervals and a number of columns equal to a user-defined number of candidate methylation meta scores.

7. The method of claim 1, wherein the aggregated methylation meta score matrix comprises a number of rows equal to the total number of candidate genomic intervals in the plurality of candidate genomic intervals and a number of columns equal to a user-defined number of candidate methylation meta scores.

8. The method of claim 1, wherein the clinical annotation data comprises patient gender, age, smoking status, disease type, disease stage, sample type, lab protocol for sample collection and processing, lab protocol for performing methylation sequencing, clinical data for treatment outcomes, or any combination thereof.

9. The method of claim 1, wherein the weighted methylation score assigned to each candidate genomic interval of the plurality of candidate genomic intervals is calculated based on a comparison of a methylation metric for each candidate genomic interval for a first subset of the plurality of patient samples to that for each candidate genomic interval for a second subset of the plurality of patient samples.

10. The method of claim 9, wherein the methylation metric comprises a CCMF signal-to-noise ratio calculated for each candidate genomic interval for the first and second subsets of the plurality of patient samples.

11. The method of claim 9, wherein the first subset of the plurality of patient samples comprises patient samples from patients diagnosed with a first disease or wherein the second subset of the plurality of patient samples comprises control samples from patients diagnosed with the first disease or wherein the second subset of the plurality of patient samples comprises patient samples from patients diagnosed with a second disease.

12. The method of claim 1, further comprising selecting a subset of the plurality of candidate genomic intervals based on the weighted methylation score assigned to each candidate genomic interval and modifying the second matrix to reduce the number of rows accordingly.

13. The method of claim 1, further comprising using the identified optimal methylation meta score to detect disease in a subject based on methylation sequencing data derived from a sample from the subject.

14. The method of claim 1, further comprising using the identified optimal methylation meta score (1) to predict a disease tissue of origin (TOO) in a subject based on methylation sequencing data derived from a sample from the subject, (2) to select a disease treatment for a subject based on methylation sequencing data derived from a sample from the subject, (3) to predict a disease treatment outcome for a subject based on methylation sequencing data derived from a sample from the subject, or (4) to identify a subject for inclusion in a clinical trial for a disease treatment based on methylation sequencing data derived from a sample from the subject.

15. A method for detection of disease and/or prediction of disease tissue of origin (TOO), the method comprising:
receiving, at one or more processors, methylation sequencing data derived from a sample from a subject;
determining, using the one or more processors, a methylation score for each genomic interval in a predetermined set of genomic intervals represented in the methylation sequencing data;
providing, to the one or more processors, the methylation scores for the predetermined set of genomic intervals as input to a trained machine learning model configured to predict a presence of disease and/or a disease tissue of origin (TOO) based on a methylation meta score derived from the methylation scores for the predetermined set of genomic intervals; and
outputting, from the one or more processors, a prediction of a presence of disease and/or a disease tissue of origin (TOO) for the subject.

16. The method of claim 15, wherein the methylation score comprises a methylation fraction score, a Cluster Consensus Methylation Fraction (CCMF) score, an unmethylated fraction score, or a Cluster Consensus Unmethylated Fraction (CCUF) score.

17. The method of claim 15, wherein the predetermined set of genomic intervals is identified based on a comparison of the methylation score calculated for each genomic interval represented in the methylation sequencing data to a methylation score threshold.

18. The method of claim 15, wherein the methylation meta score comprises an algebraic combination of the methylation scores determined for the predetermined set of genomic intervals and/or tree-based models to determine methylation scores for specific genomic intervals and/or a number of sequence reads that map to each genomic interval of the predetermined set of genomic intervals.

19. The method of claim 15, wherein the trained machine learning model is configured to output the methylation meta score, and wherein the method further comprises using the methylation meta score (1) to select a disease treatment for the subject, (2) to predict a disease treatment outcome for the subject, or (3) to identify the subject for inclusion in a clinical trial for a disease treatment.

20. A system comprising:
one or more processors; and
a memory communicatively coupled to the one or more processors and configured to store instructions that, when executed by the one or more processors, cause the system to:
receive, at one or more processors, methylation sequencing data derived from a sample from a subject;
determine, using the one or more processors, a methylation score for each genomic interval in a predetermined set of genomic intervals represented in the methylation sequencing data;
provide, to the one or more processors, the methylation scores for the predetermined set of genomic intervals as input to a trained machine learning model configured to predict a presence of disease and/or a disease tissue of origin (TOO) based on a methylation meta score derived from the methylation scores for the predetermined set of genomic intervals; and
output, from the one or more processors, a prediction of a presence of disease and/or a disease tissue of origin (TOO) for the subject.
